(19) United States
(12) Patent Application Publication
(10) Pub. No.: US 2006/0160095 A1
(43) Pub. Date: Jul. 20, 2006

(54) Survivin

(75) Inventors: Ian Hayes, Cork (IE); Tom Cotter, Cork (IE); Liam Seery, Cork (IE); Finbar Murphy, Cork (IE); Frank Altznauer, Bern (CH); Uwe Zangemeister-Wittke, Zurich (CH); Hans-Uwe Simon, Bern (CH)

Correspondence Address: PALMER & DODGE, LLP;KATHLEEN M. WILLIAMS, 111 HUNTINGTON AVENUE, BOSTON, MA, 02199, US

(73) Assignees: EiRx Therapeutics Limited; University of Bern; University of Zurich

(21) Appl. No.: 10/972,576

(22) Filed: Oct. 25, 2004

Related U.S. Application Data
(63) Continuation of application No. PCT/GB03/01753, filed on Apr. 23, 2003.
(60) Provisional application No. 60/433,126, filed on Dec. 13, 2002.

(51) Int. Cl. A61K 48/00; C12Q 1/68; G01N 33/574

(52) U.S. Cl. 435/6; 435/7.23; 514/44

(57) ABSTRACT

The invention provides a method for detecting apoptosis in a myeloid cell comprising detecting an alteration in any one of: i) a Survivin polypeptide having an amino acid sequence as set out in SEQ ID NO: 1; ii) a polypeptide having at least 80% homology with i); iii) a nucleic acid encoding a polypeptide having the sequence set out in i) or ii); iv) a nucleic acid which hybridises under stringent conditions to the sequence set out in iii); or v) the complement of iii) or iv). The invention accordingly provides a method of modulating apoptosis in neutrophils by modulating Survivin gene expression and a method of treating inflammatory disease by modulating Survivin gene expression or function.

### RELATED APPLICATIONS

This application is a continuation of International Application No. PCT/GB03/01753, which designated the United States and was filed on Apr. 23, 2003, published in English, which claims the benefit of U.S. Provisional Application No. 60/357,190, filed on Apr. 24, 2002 and U.S. Provisional Application No. 60/433,126, filed Dec. 13, 2002. The entire teachings of the above application(s) are incorporated herein by reference.

### FIELD OF THE INVENTION

The present invention relates to the use of the gene Survivin in the detection and modulation of apoptosis in terminally differentiated cells of the myeloid lineage (neutrophils, eosinophils and macrophages). In particular, it relates to a method for modulating Survivin gene expression and thus modulating apoptosis in these cells.

### BACKGROUND TO THE INVENTION

Programmed cell death or apoptosis is a genetically programmed process by which cells die under both physiological and a variety of pathological conditions (Kerr et al, Br. J. Cancer, 26, 239-257, 1972). It serves as the counter-balancing force to mitosis during adult life and is a major contributor to the sculpting of physiological structures during the many processes of development (Wyllie et al, Int. Rev. Cytol, 68, 251-305, 1980). It is characterised by a number of well-defined biochemical hallmarks. These include DNA fragmentation, caused by the activation of an endogenous endonuclease enzyme (Wyllie, Nature, 284, 555-556,1980; Enari et al., Nature, 391, 43-50, 1998). The result is a DNA ladder pattern that can be readily visualised in agarose cells. Coupled with DNA fragmentation is cell shrinkage (Wesselbory et al., Cell Immunol. 148, 234-41, 1993) where water is actively extruded from the cell. The apoptotic cell then undergoes fragmentation into apoptotic bodies that are engulfed by neighbouring cells or cells of the reticuloendothelial system.

A second well-defined characteristic is the exposure of the phospholipid phosphatidylserine to the outside surface of the plasma membrane of the cell as it undergoes apoptosis (Fadok et al., J Immunol. 148, 2207-16, 1992). Normally this lipid is located on the inner side of the membrane lipid bilayer. The underlying mechanism responsible for this lipid flipping is poorly understood at present. Its expression serves as a signal for the recognition and phagocytosis of the apoptotic cell (Fadok et al., J Immunol. 148, 2207-16, 1992)

Under normal physiological conditions apoptosis is tightly regulated. However, there are a number of diseases where the process becomes deregulated, leading to a particular pathology. Examples of where apoptosis is retarded or inhibited include some types of tumour development, a number of inflammatory conditions such as acute respiratory distress syndrome (ARDS) and other related conditions (Matute-Bello et al, Am J Respir Crit Care Med. 56, 1969-77, 1997). Inappropriate or excessive apoptosis occurs under conditions of ischaemia (stroke, myocardial infarction, etc) Linnik et al., Blood. 80, 1750-7, 1992, Gorman et al., J Neurol Sci. 139, 45-52, 1996) a series of neurodegenerative conditions, myelosuppression (Mori et al, Blood. 92, 101-7, 1998) following chemotherapy or irradiation (Lotem et al., Blood. 80, 1750-7, 1992) and a significant number of other diseases where cell death is a key feature of the pathology.

The number of factors which are know to induce survival in particular cell types is ever increasing (e.g. IL2, IL3, IL4, IL5, IL8, GM-CSF, insulin like growth factor 1, NGF, VEGF, PDGF, SCF, LIF, EGF etc.). Many of these survival factors appear to share a commonality in the survival pathway (Datta S R et al. Genes and Development 13: 2905-2927, 1999). For an extracellular stimuli, to confer survival on a cell, it must inhibit the endogenous apoptotic machinery. The model predicts that there is a series of temporal events that occur upon survival factor/ receptor interaction. The first of these is tyrosine phosphorylation at the plasma membrane due either to intrinsic receptor tyrosine kinase activity (e.g. the insulin growth factor 1 receptor), or indirectly coupled to tyrosine kinases or alternatively directly coupled to several transmembrane G protein-coupled receptors.

Haematopoietic blood cells are derived from pluripotent bone marrow stem cells by proliferation and differentiation. The stem cells, which are characterised by their repertoire of cell surface receptors (e.g. CD34+ and Lin−), are undifferentiated. Through rounds of self-renewal and expansion by proliferation, followed by differentiation in response to cytokines and growth factors, these cells give rise to all of the mature functional blood cells (monocyte/macrophages, eosinophils, neutrophils, T lymphocytes and B lymphoctes).

In the differentiation of blood cells, two lineage pathways are clearly delineated; myeloid (including monocyte/macrophages, eosinophils and neutrophils) and lymphoid (including T lymphocytes and B lymphoctes). In general terms, myeloid cells perform functional roles associated with the rapid killing and removal of pathogens (such as bacteria and parasites) while the lymphoid cells are associated with the memory of pathogens and the generation of antibody-mediate immune response. Myeloid cells perform a more transient function and so are produced in large numbers, survive days/weeks rather than months or years, and are fully and terminally differentiated, functional specialised cells that do not divide but remain in the GO/GI phase of the cell cycle. Once they have performed their function they die by apoptosis and are removed by phagocytosis. In contrast, and because of their specialised memory role T and B lymphocytes can be long lived and are capable of extensive proliferation and cell cycle.

Blood neutrophils are myeloid cells having relatively short lives with greater than 80% of them apoptosing within the first 24 hours. Apoptotic neutrophils are phagocytosed by macrophages via thrombospondin and macrophage CD36/vitronectin receptor (Savil 1992, Clinical Science 83, 649-55, Savil et al. 1993, Immunology Today 14,131-136) and thus prevent release of a potentially lethal cocktail of enzymes in the host, should the neutrophil undergo necrosis. However, certain inflammatory environments favour the survival of neutrophils. In vitro, several cytokines including GM-CSF, IL-1, IL-2, IL-8 and IFNγ can delay neutrophil apoptosis (Brach et al, 1992, Blood 80, 2920-2924; Calotta et al 1992, Blood 80, 2012-2020, Lee et al 1993, J Leuk Biol 54, 283-388, Pericle et al 1994, Eur. J. Immunol24, 440-444, Get ref for IL8). Cytokine fluids such as Bronchoalveolar lavage (BAL) obtained from the lungs of disease patients has also been shown to increase the survival in culture of isolated peripheral blood neutrophils. Furthermore, inflammatory proteins (e.g. C5A) and bacterial products (e.g. LPS) have also been shown to inhibit apoptosis. These findings together with other results demonstrating that the presence of either actinomycin D or cycloheximide can promote apoptosis in PMN (Whyte et al, 1991, Clin Sci. 80:5p) suggests a role for active gene expression and translation in control of PMN apoptosis. Moreover, other investigators have shown that NFκB regulated genes seem to play a critical role in preventing apoptosis induced by TNFα, since inhibition of this transcription factor using the fungal metabolite Gliotoxin, induces rapid apoptosis (Ward et al. 1999, J. Biol. Chem. 274. 4309-4318). The same investigators also demonstrated that blocking NFκB with Gliotoxin removes the anti-apoptotic effect of LPS. Yoshida et al have identified an alternative mode of action of gliotoxin. These investigators demonstrated that gliotoxin inhibited NADPH oxidase and consequently prevented the onset of superoxide generation by human neutrophils in response to phorbol myristate. (Yoshida et al Biochem Biophys Res Commun 2000, 268(3) 716-23).

Granulocyte macrophage colony-stimulating factor (GM-CSF) is known to inhibit PMN apoptosis both in vitro and in vivo (Cox et al. 1992, Am. J. Respiratory Cell Mol Biol. 7, 507; Chintinis et al 1996, J. Leuk Biol 59:835). One consequence of GM-CSF treatment of PMN is a time and dose dependent tyrosine phosphorylation event within the cell (McCall et al., 1991, Blood 78(7) 1842-52). That tyrosine phosphorylation is implicated in the regulation of apoptosis has been demonstrated (Simon et al 1995, Int. Arch Allergy Immunol. 107, 338-339). These workers demonstrated that the effect of GM-CSF on granulocyte cell death could be attenuated by the tyrosine kinase inhibitor genestein, suggesting that increases in tyrosine phosphorylation are essential to inhibit cell death. To further analyse a role for tyrosine phosphorylation, the authors increased levels of tyrosine phosphorylation using the protein-phosphatase inhibitor phenylansine oxide (PAO). Similar to GM-CSF, treatment of the cells with PAO is followed by a large increase in tyrosine phosphorylation and matched inhibition of apoptosis. Inhibitors of tyrosine phosphorylation (Genestein and Herbimycin A) reversed the effects of PAO on tyrosine phosphorylation and neutrophil apoptosis.

Furthermore, Wei et al (J. Immunology 1996,157, 5155-5162) suggested specificity in the anti-apoptotic signalling pathway by showing that GM-CSF inhibition of programmed cell death did not appear to be related to known proteins associated with cell survival i.e. p53, cdc2, Rb, and Bcl-2. However GM-CSF did induce a rapid activation of Lyn, a src family tyrosine kinase, and Lyn antisense treatment of neutrophils reversed the survival promoting effect of GM-CSF. Other investigators have demonstrated that GM-CSF selectively induced tyrosine phosphorylation of Extracellular Signal-Related kinase (ERK), a member of microtubule associated protein kinase (MAPK) family (Yuo et al. 1997, BBRC 235, 42-46). Al-Shami et al. (Blood 1997, 89(3) 1035-1044) has shown that GM-CSF induces both a time and concentration-dependent increase in the level of tyrosine phosphorylation of the PI-3-kinase regulatory subunit p85, possibly via lyn kinase. In corroboration of these results, Klein et al. (J. Immunol. 2000, 164, 4286-4291), using pharmacological inhibitors of signal transduction, further demonstrated a role for PI 3-kinase and ERK. These investigators showed that GM-CSF caused a rapid phosphorylation of the protein Akt, a substrate for PI 3-kinase. Akt phosphorylation is in turn associated with phosphorylation of BAD, a pro-apoptotic member of the Bcl-2 family. The authors hypothesised that this phosphorylation resulted in disengagement of Bad with anti-apoptotic family members of Bcl-2 family, allowing them to prevent neutrophil apoptosis.

The link between GM-CSF and tyrosine phosphorylation and inhibition of programmed cell death has until recently been unknown. Previously, prolonged survival of PMN caused by inhibition of apoptosis is observed in bcl-2 transgenic mice (Lagasse and Weissman, 1994, J. Exp. Med. 179 1047). This result is surprising since normal peripheral blood neutrophils are negative for bcl-2 (Wei et al. J. Immunology 1996,157, 5155-5162), however it does show that targets for the Bcl-2 family of apoptosis associated proteins can control PMN apoptosis. Weinnman et al. (1999, Blood, 93, 3106-3115) investigated the role of other members of the Bcl-2 family in regulating PMN apoptosis. The authors cultured PMN for 0, 2,6 or 22 h in the presence of TNFα (pro-apoptotic) or GM-CSF or are left untreated. Fresh, unstimulated PMN showed a high level of expression of Bcl-XL that gradually decreased as the culture proceeded, suggesting that loss of this protective protein may play a role in spontaneous apoptosis. The reduction of Bcl-XL in the presence of TNFα is much stronger when compared to control cells. GM-CSF did not alter the effect of Bcl-XL. Next the investigators examined expression of Bax-α, a proapoptotic member of the Bcl-2 family. Results showed that GM-CSF induced a down regulation of Bax-α when compared to control cells, suggesting that the down-regulation of this death promoting is involved in PMN survival mediated by GM-CSF. The authors concluded that GM-CSF seems to promote survival by modulating the Bax-α/Bcl-XL ratio via down regulation of Bax-α. Furthermore, the authors suggested that inhibition of apoptosis by GM-CSF might be due to a caspase 3 regulation since no further reduction of apoptosis is observed, above that already seen, when PMN are stimulated GM-CSF after inhibition of caspase-3 with its inhibitor Z-DEVD-FMK.

Other members of the Bcl-2 family have also been implicated in neutrophil apoptosis. Expression of myeloid cell leukaemia 1 (MCL1), another viability-promoting family member, has been shown to decrease during neutrophil apoptosis but increases in response to GM-CSF and LPS, suggesting a link with PMN survival, (Moulding D A, Quayle J A, Hart C A, Edwards S W, Blood 1998; 92(7): 2495-502). Neutrophils also express mRNA for A1, another Bcl-2 homologue with anti-apoptotic properties (Chuang P I, Yee E, Karsan A, Winn R K, Harlan J M, Biochem Biophys Res Commun 1998; 249(2): 361-5). The authors demonstrated that agonists that promote cell survival (e.g. LPS and G-CSF) up-regulated the message for this protein. Moreover, neutrophil apoptosis is enhanced in mice that lack A1-a, a subtype of the A1 gene, and LPS-induced inhibition of apoptosis is abolished. However in these mice TNFα induced apoptosis is unchanged, which suggest that A1 is involved in regulating some but not all neutrophil apoptotic pathways (Hamasaki A, Sendo F, Nakayama K, Ishida N, Negishi I, Nakayama Ki, Hatakeyama S, J Exp Med 1998; 188(11):1985-92).

In our copending international patent application, WO 02/04657, we have shown that GM-CSF inhibits death through apoptosis by the regulation of ‘effector genes’ that control the process of apoptosis. A signal acts through a signal transduction cascade and is associated with significant changes, or patterns of changes, in gene expression in the cell. To date, however, the identities of such ‘effector genes’ and their role in the signalling pathways that lead to the biochemical events of cell death have been incompletely determined.

Our previous work, as described in WO 01/46469 and WO 02/04657, therefore establishes two assays for the identification of genes involved in the regulation of apoptosis, using the neutrophil as a model, by screening for genes whose expression is modulated by changes in intracellular ROS concentrations and/or the action of GM-CSF. Using these assays, we have verified changes in expression in a number of known genes whose role in apotosis has been previously established—such as various Bcl-2 related proteins and caspases. The assays therefore provide a method for validating the involvement of candidate genes in apoptosis and, in particular, myeloid cell apoptosis.

The control of apoptosis represents a significant therapeutic target, since many diseases are due to defects in this process. Many physiological factors induce and prevent cell apoptosis. For example, cytokines or growth factors such as GM-CSF inhibit death through apoptosis. There is an acute need to identify the genes that regulate this process. In other words, if one identifies a gene that prevents apoptosis in a particular cell, then this gene/gene product or its function can be blocked by a drug and apoptosis in that cell allowed to occur.

A number of inflammatory diseases such as asthma, chronic obstructive pulmonary disease (COPD), Cystic Fibrosis (CF), Rheumatoid Arthritis (RA) and Inflammatory bowel disease (IBD) are characterised by a) elevated levels and expression of cytokines and growth factors that act predominantly on myeloid cells, b) prolonged survival of myeloid cells, and c) prolonged activation of myeloid cells.

Increased numbers of activated myeloid cells such as neutrophils are associated with, and strongly implicated in, the pathology of a number of these chronic and acute inflammatory diseases (Williams T J and Jose P J: Novartis Found Symp 2001;234:136-41; discussion 141-8; Barnes P J: Chest 2000 February;117(2 Suppl):10S-4S; Nadel J A: Chest 2000 February;117(2 Suppl):10S-4S; Ward I et al: Trends Pharmacol Sci 1999 December;20(12):503-9; Bradbury J and Lakatos L: Drug Discov Today 2001 May 1;6(9):441-442).

The increased levels of cytokines and growth factors are thought to promote the differentiation, survival and activation of myeloid cells. Such cytokines and growth factors include GM-CSF, IL-8 and IL-5 (each of which act through IL-3 family receptors), IL-8, TNFalpha and G-CSF. GM-CSF, G-CSF, IL-8 and TNF alpha have been shown to increase the survival and/or recruitment and activation of peripheral blood neutrophils (Matute-Bello G et al: Crit Care Med 2000 January; 28 (1): 1-7; Eidelman O et al: Mol Med 2001 August; 7 (8):523-34; Tagg Physiol Lunc Cell Mol Physiolo 2000, January; 278 (1): L33-41; Noguchi M et al: Digestion 2001; 63 Suppl 1:32-6; Ina K et al: Gastroenterol Hepatol 1999 Jan; 14 (1): 46-53 and Fanning N F et al: Shock 1999 March;11(3):167-74) in chronic inflammatory disease. Examples include both the use of individual cytokines, but also the more disease relevant use of cytokine fluids such as Brochoalveolar lavage (BAL) obtained from the lungs of disease patients. For example, BAL can be shown ex-vivo to increase the survival in culture of isolated peripheral blood neutrophils (see, for example, Reynolds H Y; Lung 2000, 178(5):271-293).

Delayed apoptosis of terminally differentiated cells of the myeloid lineage, neutrophils, eosinophils and macrophages in-vivo has been demonstrated to be associated with inflammatory diseases (Watson R W et al: Surgery 1997; August 122(2):163-71; Brannigan A E et al: Shock 2000; May 13(5):361-6; Vandivier R W et al: J. Clin Invest 2002 March 109(5):661-70; Oberholzer C et al: Proc Natl Acad Sci USA. 2001 Sep. 25;98(20):11503-8; Zangemeister-Wittke U and Simon H U: Cell Death Differ 2001 May;8(5):537-44; Simon H U: Immunol Rev 2001 February;179:156-62; Heinisch I V et al: Eur J Immunol 2000 December;30(12):3441-6 and Dibbert B et al: Proc Natl Acad Sci USA 1999 Nov. 9;96(23):13330-5). Indeed eosinophils and macrophages both respond to many of the same cytokines as neutrophils, with increased survival and activation. Cytokines of the IL-3 family demonstrate particular potency towards each of these cells.

It is an accepted therapeutic approach for such diseases to identify drugs (and drug targets) that would inhibit the survival, activation and/or recruitment of neutrophils (or other inflammatory cells). For example, steroids are used to very effectively treat chronic inflammation associated with asthma. Steroid action is mediated, at least in part, by inhibiting eosinophil survival and activation in response to cytokines present in the disease fluids. Neutrophils have also been shown, using a transgenic in-vivo model, to be a critical component of Rheumatoid arthritis pathology. The removal of systemic neutrophils using antibodies, significantly reduced joint inflammation (Wipke B T and Allen P M: J Immunol 2001 Aug. 1;167(3):1601-8). Similarly, the efficacy of Cilomilast in treating COPD may rest with its ability to reduce neutrophil inflammation (Hele D J: Meeting Report from 2001 American Thoracic Society Meeting http://respiratory-research.com/content/2/5/E003). Furthermore, induction of neutrophil apoptosis, in an in-vivo model of acute lung inflammation, using aerosolised opsonized dead E.coli, significantly improved lung injury parameters (Sookhai et al; Ann Surg 2002 February 235 (2):285-91).

However, inflammatory diseases are generally poorly treated, and it would be very desirable to identify additional drugs that prevent the survival and/or activation of one or more of the myeloid cells in inflammation. It is therefore desirable to identify survival factors expressed by myeloid cells that confer this prolonged survival.

Survivin is a 142 amino acid protein (approx. 16.5 kDa) that is expressed in tumor cells and embryonic tissues (Adida et al. (1998) Am. J. Pathol. 152 (1):43-9; and (1997) Gastroenterology 113 (4):1060). The gene is located on chromosome 17q25 and is a novel member of the IAP family of apoptosis inhibitors. The nucleic acid which encodes Survivin is related to that of Effector Cell Protease Receptor-1 (EPR-1) but its orientation is assigned to the antisense EPR-1 strand.

In contrast to other members of the IAP family which are widely expressed in human tissues, Survivin is expressed primarily in fetal but not adult tissues. Moreover, while dramatic overexpression of Survivin is found in most cancers (Ambrosini et al. Nat. Med. 1997; 3:917-921) expression has been found to be undetectable in most terminally differentiated normal tissues.

Survivin is the only IAP whose expression is cell cycle dependent. In fact, Survivin expression is required for proper execution of mitosis and cell division (Reed, 1999, Cell. 102;545-548) and this is confirmed by the phenotype of knockout mice suggesting a critical role for Survivin in mitosis.

It has been proposed that the enhanced expression of Survivin in transformed cells promotes cell survival through binding to the mitotic spindle during G2/M phase of the cell cycle (Li et al. Nature; 396; 580-583) and that this counteracts the default induction of apoptosis at this stage. Overexpression may overcome the G2/M phase checkpoint to enforce progression of cells through mitosis. The survival effect combined with the aberrant expression of Survivin in dividing cancer cells has lead to the development of therapeutic strategies for cancer aimed to induce apoptosis by modulating Survivin expression.

### SUMMARY OF THE INVENTION

The present invention identifies Survivin as a key regulator of apoptosis/survival in non-dividing cells of the myeloid lineage, with particular reference to the neutrophil. In particular, Survivin gene expression is decreased in neutrophil apoptosis and increased in neutrophil survival when apoptosis is inhibited by the presence of GM-CSF. Furthermore, when GM-CSF inhibition of apoptosis is blocked by gliotoxin, Survivin expression is down regulated. In addition, decreased expression of recombinant Survivin in neutrophils by introducing antisense RNA resulted in significant inhibition of the survival effect of haematopoeitins such as those present in BAL from chronic inflammatory diseases

This finding was wholly unexpected, given the previous association of Survivin with proliferating cells, especially tumour and foetal cell types, and the functional association of Survivin with the G2/M phase of the cell cycle. In contrast to tumour cells, neutrophils are terminally differentiated cells arrested in a quiescent G0/G1 stage. A role for Survivin in cell survival in non-dividing cells has not previously been identified. In fact, and in contradiction to the observations reported herein, a previous study of Redox-regulated neutrophil apoptosis failed to show any increase in Survivin gene expression in the presence of GM-CSF (Watson: Antioxidants and Redox Signalling, 2002, 4(1):97-104). Many inflammatory diseases are mediated by differentiated cells of the myeloid lineage. The therapeutic modulation of Survivin activity in these cells will therefore fill a significant unmet clinical need.

The amino acid sequence for Survivin is set out in SEQ ID NO: 1 and identified in the GenBank protein database under accession number AAC51660.

| SEQ ID NO:1 | |
|---|---|
| 1 | MGAPTLPPAW QPFLKDHRIS TFKNWPFLEG CACTPERMAE AGFIHCPTEN EPDLAQCFFC |
| 61 | FKELEGWEPD DDPIEEHKKH SSGCAFLSVK KQFEELTLGE FLKLDRERAK NKIAKETNNK |
| 121 | KKEFEETAKK VRRAIEQLAA MD |

The identification and role of this gene in neutrophil apoptosis has been validated using model assays described in our copending applications WO 01/46469 and WO 02/04657 as described herein.

These model discovery assays are configured to target the ‘early’ regulatory events occurring in apoptosis induced by ROS and, in particular, in the inhibition of apoptosis by GM-CSF. When apoptosis by GM-CSF is itself inhibited by a drug, such as gliotoxin, then changes, or patterns of changes can be targeted by clustering those changes that are common and both increase and/or decrease depending on the treatment. For example, a change that is a ‘decrease’ following induction of apoptosis is a candidate target gene, however, a change that is additionally an ‘increase’ following inhibition of apoptosis by GM-CSF has a higher probability of being a target gene because its regulation shows increased correlation with the process. Likewise, a change that is further a ‘decrease’ following inhibition of GM-CSF inhibitory effect has a yet higher probability of being a target gene because its regulation shows increased correlation with the process.

Genes regulated in these models following modulation of apoptosis include genes that 1) are ‘effector’ genes involved in the cells defence mechanisms aimed at preventing apoptosis (anti-apoptotic genes) and thus represent therapeutic targets, 2) make up aspects of the apoptosis and/or GM-CSF signal cascade and thus represent therapeutic targets, 3) initiate the process of apoptosis (pro-apoptotic genes) and thus represent therapeutic targets, and 4) are associated with the processes of apoptosis and defence that will aid in the understanding of key pathways, processes and mechanisms that may subsequently lead to the identification of therapeutic targets.

We have previously demonstrated that these cell-based apoptosis models, which are combined with a genomics approach, identify genes known to be involved in apoptosis and defence. In these models, the expression of Survivin correlates with that of known apoptosis genes, redox modulation and survival genes thus confirming its role in apoptosis or in modulating apoptosis in neutrophils. We therefore provide a method for detecting apoptosis in neutrophils comprising detecting a decrease in Survivin gene expression.

Accordingly in a first aspect of the invention, there is provided a method for detecting apoptosis in a myeloid cell comprising detecting a decrease in any one of:
i) a Survivin polypeptide having an amino acid sequence as set out in SEQ ID NO: 1;
ii) a polypeptide having at least 80% homology with i);
iii) a nucleic acid encoding a polypeptide having the sequence set out in i) or ii);
iv) a nucleic acid which hybridises under stringent conditions to the sequence set out in iii); or
v) the complement of iv).

In another aspect of the invention there is provided a method for detecting survival in a myeloid cell comprising detecting an increase in Survivin activity or expression by detecting an increase in any one of:
i) a Survivin polypeptide having an amino acid sequence as set out in SEQ ID NO: 1;
ii) a polypeptide having at least 80% homology with i);
iii) a nucleic acid encoding a polypeptide having the sequence set out in i) or ii);
iv) a nucleic acid which hybridises under stringent conditions to the sequence set out in iii); or
   the complement of iii) or iv).

In the context of the present invention, the term “myeloid cell” refers to terminally differentiated, non-dividing cells of the myeloid lineage. These cells include neutrophils, eosinophils and monocytes/macrophages. In one embodiment of any aspect of the present invention, the myeloid cell is a neutrophil, eosinophil or monocyte/macrophage.

In one embodiment, the polypeptide is a splice variant of surivin. In particular, the splice variant is selected from the group consisting of Survivin delta ex3 and Survivin 2B.

Levels of gene expression may be determined in any appropriate manner. Detecting a decrease or increase in gene expression may be achieved by measuring Survivin gene expression in treated versus non-treated cells. Preferably, gene expression may be measured by detecting nucleic acid encoding a Survivin polypeptide such as Survivin mRNA transcripts, or a fragment thereof. In one embodiment, the method of measuring mRNA transcripts may use an amplification technique as described herein. In another embodiment, Survivin expression may be measured by detecting the Survivin polypeptide gene product, or fragment thereof, using, for example, agents that bind Survivin. Suitable agents include anti-Survivin antibodies.

In another aspect, there is provided a method of detecting GM-CSF-induced cell survival by detecting an increase in Survivin gene expression.

In another aspect, there is provided a method of modulating apoptosis in a myeloid cell comprising the step of increasing, decreasing or otherwise altering the functional activity of Survivin or the nucleic acid encoding it. In one embodiment, said modulation is to increase apoptosis. In another embodiment, said modulation of apoptosis decreases survival in a myeloid cell.

In the context of the present invention the term ‘altered functional activity of Survivin or the nucleic acid encoding it’ includes within its scope increased, decreased or an otherwise altered activity of Survivin as compared with the native protein functioning in its normal environment, that is within a single cell under native conditions. In one embodiment, said cell is a neutrophil. In addition, it also includes within its scope an increased or decreased level of expression and/or altered intracellular distribution of the nucleic acid encoding Survivin, and/or an altered intracellular distribution of Survivin itself.

In one embodiment, the method of modulating apoptosis involves decreasing Survivin gene expression. In a preferred aspect, the expression of Survivin is reduced by greater than 50%, 60%, 70%, 80%, 90% or more of its normal level in untreated cells.

Preferably, a decrease in Survivin gene expression may be effected by antisense expression. Other means of decreasing Survivin gene expression will be recognised by those skilled in the art and include introducing dominant negatives, peptides or small molecules.

Suitable antisense molecules include those described, for example, in Olie et al. Cancer Research 60, 2805-2809 and referred to herein as “4003”. In one embodiment, the antisense molecules are chosen so as to inhibit expression of Survivin or any variants thereof including splice variants. Suitable antisense molecules may be oligonucleotides comprising natural nucleic acids or may be variants, based on these molecules, which have been chemically modified. A number of chemical modifications to oligonucleotides, such as antisense molecules, are known and are used to increase the effects of such molecules. Suitable modifying chemistries include, without limitation, the generation of phosphorothioate, methylphosphonate and locked nucleic acid derivatives as well as 2′-O-methoxy-ethoxy (2′-MOE) treatment, to generate gapmer versions of 4003.

In a further aspect, there is provided the use of Survivin, or an agent that alters Survivin expression in a cell, in the modulation of myeloid cell apoptosis.

The induction of myeloid cell apoptosis is desirable as a therapeutic treatment for conditions characterised by abnormal persistence of terminally differentiated myeloid cells including neutrophils, eosinophils and monocytes/macrophages. Such conditions include disease characterised by chronic or acute inflammation such as chronic inflammatory disorders for example, cystic fibrosis, acute respiratory distress syndrome, chronic obstructive pulmonary disease, inflammatory bowel disease and rheumatoid arthritis.

Accordingly in another aspect of the invention there is provided a method of treatment of inflammatory disease comprising administering a modulator of Survivin gene expression or functional activity.

In another aspect, there is provided the use of a modulator of Survivin expression or activity in the manufacture of a medicament for use in the treatment of inflammatory disease.

Inflammatory diseases include, but are not limited to, diseases such as sepsis, Acute Respiratory Distress Syndrome, Pre enclampsia, Myocardial ischemia, reperfusion injury, Psoriasis, Asthma, COPD, bronchiolitis, Cystic Fibrosis, Rheumatoid Arthritis, Inflammatory Bowel Disease, Crohns Disease and Ulcerative colitis.

In one embodiment of either of these aspects, said modulator is a modulator of Survivin gene expression and, in particular, an antisense RNA molecule. In a preferred embodiment, the antisense molecule is a 20-mer phosphorothioate antisense oligonucleotide. In a particularly preferred embodiment, the antisense molecule targets nucleotides 232-251 of Survivin mRNA. Other suitable antisense molecules for decreasing Survivin expression are described in U.S. Pat. No. 6,335,194.

In another embodiment, the modulator of Survivin activity is a modulator compound which interacts with Survivin cellular activity. This interaction can be either direct interaction with Survivin or indirect interaction e.g. through the modulation of the activity of a regulator of Survivin function or stability. For example, cdc2 kinase inhibitors such as flavopiridol are expected to inactivate Survivin by preventing its phosphorylation.

In another embodiment, the modulator is a Survivin binding compound such as an antibody. Suitable, anti-Survivin antibodies are described, for example, in Fukuda et al. (Blood, 2001, 98: 7; p.2091-2100).

Survivin may itself be used to identify other candidate genes or proteins which are involved in myeloid cell apoptosis.

Accordingly, in a further aspect, there is provided a method for identifying a molecule which interacts with Survivin in myeloid cell apoptosis.

In the context of the present invention, molecules which ‘interact’ with Survivin include molecules which bind to Survivin either directly or indirectly. Methods for detecting those molecules include physical methods and molecular biology techniques as herein described. Suitable standard laboratory techniques will be familiar to those skilled in the art and include immunoprecipitation, immunoblotting and fluorescence techniques. One skilled in the art, will appreciate that this list is not intended to be exhaustive. Suitable molecular biology techniques include phage display and the yeast two-hybrid system described herein.

Another aspect of the invention is directed to the identification of agents capable of modulating Survivin gene expression or protein function in myeloid cells. In this regard, the invention provides assays for determining compounds that modulate the function and/or expression of Survivin.

### BRIEF DESCRIPTION OF THE DRAWINGS

FIG. 1 Measurement of Apoptosis by DNA fragmentation. Neutrophils are cultured for 18 h in the presence (+, 0.5units/ml) or absence (−) of GM-CSF (5U), and then analyzed for apoptosis by measurement of DNA fragmentation. G0/G1 and sub-G1 cells are indicated.

FIG. 2 shows the dose responsiveness of the anti-apoptotic effect of GM-CSF. Optical densities are read at 570 nm using a plate reader. The results indicate a direct correlation between survival and concentrations of GM-CSF added to the culture medium.

FIG. 3 shows that the fungal metabolite gliotoxin blocks the GM-CSF in the inhibition of neutrophil apoptosis. The method is as described in Example 1. Optical densities are read at 570 nm using a plate reader. Gliotoxin effectively blocks the GM-CSF mediated inhibition of neutrophil apoptosis. The blocking effect is not seen when the inactive analogue of gliotoxin, methylgliotoxin is added with GM-CSF. No increased neutrophil apoptosis is seen with the addition of gliotoxin alone to isolated neutrophils, demonstrating that the effect is specific to, and limited to, a reversal of the protective effects of GM-CSF.

FIG. 4 shows the gel image of a microarray.

FIG. 5 shows the analysis of a captured image film by Array Vision™ software.

FIG. 6 shows the results of combined code cluster analysis.

FIG. 7 shows cluster analysis of LifeGrid filters. Human purified peripheral blood neutrophils are either allowed to undergo spontaneous apoptosis (Apop), or else are treated with 5 U/ml GM-CSF to inhibit apoptosis (GM-CSF). Samples are isolated for RNA extraction and microarray gene analysis, 2 h (Apop2 and GMCSF2), 3 h (Apop3), 4 h (Apop4 and GMCSF4), 5 h (Apop5) and 6 h (Apop6 and GMCSF6) post-isolation. In some experiments Gliotoxin (0.1 μg/ml; Glio) or its inactive analogue Methyl Gliotoxin (0.1 μg/ml; Methyl) are added in the presence of GM-CSF. Average fold change values (from two spots on the filters) for selected candidate apoptosis/survival-associated genes are compared to time zero controls (except GM4 which compares fold change of 4 h treatment of GM-CSF plus Gliotoxin with 4 h treatment of GM-CSF with Methyl Gliotoxin control), are analysed by GeneMaths using a Pearson correlation and Ward cluster algorithms. Increased expression (light) and decreased expression (dark) are represented and referenced by a color scale bar. Survivin gene is highlighted in bold (identified by number 2504586).

FIG. 8 shows Survivin mRNA is increased in GM-CSF-induced neutrophil survival, and, this increased expression is blocked by Gliotoxin. Human purified peripheral blood neutrophils are treated as described in FIG. 6. The relative amounts of Survivin transcripts are shown.

FIG. 9 shows a dendrogram representation of cluster analysis for FIG. 6. Marker genes, with known function in apoptosis and survival are indicated. Survivin gene is highlighted in bold.

FIG. 10 shows expression of Survivin isoforms in neutrophils treated with GMCSF. Three products are detected as predicted, corresponding to Survivin-2B (498 bp), Survivin (429 bp) and Survivin-Δex3 (311 bp). A control sample from HL60 cells is also shown.

FIG. 11 shows a time course of neutrophil differentiation, measured by NBT reduction assay. HL-60 cells are seeded at 5×10⁵/ml and incubated with 10 μM Retinoic acid for 24, 48 and 72 hours, or untreated (HL60). Cells are harvested and stimulated with PMA and incubated for 15 minutes in the presence of NBT. Approximately 5×10⁴ cells are transferred by cytospin onto slides and counter stained with Eosin. Slides are analysed blind and only whole cells containing formazan deposits were considered positive. Graph demonstrates differentiation towards the neutrophil lineage, as measured by percentage NBT positive cells.

FIG. 12 shows a time course of neutrophil apoptosis, following differentiation of HL60 cells. HL60 cells are seeded at 5×10⁶cells in T25 flasks and incubated with 10 μM Retinoic acid for 24, 48, 72, 96, 120 and 144 hrs. Mock treated HL-60 cells were used as a control. Cellular DNA was analysed for fragmentation into oligonucleosomal-size fragments and their multiples by agarose gel electrophoresis. Lanes 1,3,5,7,9,11 contain DNA from HL-60 cells that were differentiated with 10 μM Retinoic Acid over the indicated period whereas samples in lanes 2,4,6,8,10,12,14 are from control HL-60 cells treated for the same time period. Lane 15 is a 1 Kb Plus Ladder Molecular Marker.

FIG. 13 shows Survivin is differentially regulated during neutrophil differentiation. HL60 cells are treated as described in FIG. 11. cDNA is hybridised on Incyte LifeGrid filters. Average fold change (from two spots on the filters) for Survivin gene on the Incyte LifeGrid filters are compared to time zero controls.

FIG. 14 shows survivin levels are markedly increased in neutrophils exposed to survival factors in vitro and in inflammatory disease neutrophils (cystic fibrosis).

(A) Real-time PCR quantification of survivin mRNA. The lung cancer cell line A549 serves as a standard (=100%). Cystic fibrosis neutrophils contain increased amounts of survivin mRNA compared to normal neutrophils (upper panel). The increase in survivin mRNA in cystic fibrosis neutrophils is mimicked by addition of the inflammatory cytokines GM-CSF and G-CSF to normal control neutrophils in vitro (lower panel). Values are means ±S.E.M. of three independent experiments. *, p<0.05; ***, p<0.001. (B) Immunoblotting. Levels of survivin in neutrophils are increased by culturing normal control neutrophils for 12 h in the presence of GM-CSF and G-CSF. In addition, freshly isolated neutrophils of patients suffering from cystic fibrosis (CF, n=4) show strongly increased amounts of survivin compared to normal control individuals (first lane). Purified recombinant survivin serves as positive control (Co). β-actin immunoblotting is performed to demonstrate equal loading.

FIG. 15 shows real-time PCR quantification of survivin mRNA in TF1 cells transfected with survivin antisense (as) and missense (ms) PT, LNA and MeP oligos. The amount of survivin transcript is expressed as a percentage of survivin detected in mock-transfected cells.

FIG. 16 shows that a reduction of survivin expression, by treatment with antisense molecules having LNA-modified chemistries, blocks the GM-CSF-mediated delay of apoptosis in neutrophils. Antisense (as) LNA oligonucleotides (closed box), targeted against survivin mRNA, increases spontaneous apoptosis of neutrophils cultured in the presence of GM-CSF assayed 18-h post transfection when compared to neutrophils transfected with mis-sense (ms) LNA oligonucleotides (open box).

FIG. 17 shows antisense treatment specifically prevents increases in survivin gene expression in neutrophils upon stimulation with hematopoietins.

(A) Real-time PCR quantification of survivin mRNA. The lung cancer cell line A549 serves as a standard (=100%). Survivin-antisense (as) is compared to mismatch control oligonucleotides (ms) in the absence of survival cytokines following a 4-h transfection period (upper panel) and upon GM-CSF (middle panel) and G-CSF (lower panel) stimulation. Values are means ±S.E.M. of three independent experiments. *, p<0.05; **, p<0.01; ***, p<0.001.

(B) Protein expression after incubation with survivin antisense oligonucleotides (as) following a 15-h transfection period (upper panel) is compared to treatment with missense (ms), upon GM-CSF (middle panel) and G-CSF (lower panel) stimulation. β-actin immunoblotting is performed to demonstrate equal loading. Results are representative of three independent experiments.

FIG. 18 shows reduction of survivin expression by specific antisense treatment completely blocks the GM-CSF- and G-CSF-mediated delay of apoptosis in neutrophils. Antisense oligonucleotides targeting survivin mRNA (as, closed symbols), are compared to control ms oligonucleotide (ms; open symbols) after a 10-h transfection period, as assessed by formation of oligonucleosomal DNA-fragments (left panel). GM-CSF (middle panel) and G-CSF (right panel) prevents DNA-fragmentation and survivin-antisense blocks GM-CSF- and G-CSF-mediated survival in a dose-dependent manner. Values are means ±S.E.M. of four independent experiments. *, p<0.05; **, p<0.01; ***, p<0.001.

FIG. 19 shows survivin-antisense treatment abolishes the inhibitory effect of neutrophil hematopoietins on caspase-3 activation in neutrophils.

(A) Immunoblotting. Apoptosis is associated with decreased levels of the 32-kDa proform of caspase-3 and with the occurrence of a 17-kDa caspase-3 fragment. The effects of Survivin-antisense treatment (as) and mismatch control oligonucleotides (ms) are compared. The same results are obtained in two additional experiments.

(B) Caspase-3 activity assay: Increases in the enzymatic activity are detectable in neutrophils undergoing spontaneous apoptosis compared to GM-CSF- or G-CSF-treated cells. The effects of Survivin-antisense treatment (as) and mismatch control oligonucleotides (ms) are compared. Results of two independent experiments are shown (circle, experiment 1; triangle, experiment 2).

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

### DEFINITIONS

“Apoptosis” or cell death is a controlled intracellular process characterised by the condensation and subsequent fragmentation of the cell nucleus during which the plasma membrane remains intact.

By “modulating apoptosis” is meant that for a given cell, under certain environmental conditions, its normal tendency to undergo apoptosis is changed compared to an untreated cell. For example, blood neutrophils have a defined apoptotic tendency—within a population of cells, greater than 80% will apoptose within the first 24 hours. Modulating the apoptosis of blood neutrophils means changing this normal apoptotic tendency such that apoptosis is increased or decreased relative to the normal rate. Similarly, blood neutrophils in the presence of GM-CSF have a decreased tendency to apoptose. Thus, modulating apoptosis of blood neutrophils in the presence of GM-CSF means increasing or decreasing apoptosis relative to their normal decreased tendency under these conditions. A decreased tendency to apoptose may also be a measurable increase in cell survival and may be the result of an inhibition of apoptosis by inhibiting one or more components of the apoptotic pathway.

The term “expression” refers to the transcription of a genes DNA template to produce the corresponding mRNA and translation of this mRNA to produce the corresponding gene product (i.e., a peptide, polypeptide, or protein). The term “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expresssion in the absence of said treatment.

“Antibodies” can be whole antibodies, or antigen-binding fragments thereof. For example, the invention includes fragments such as Fv and Fab, as well as Fab′ and F(ab′)₂, and antibody variants such as scFv, single domain antibodies, Dab antibodies and other antigen-binding antibody-based molecules.

The “functional activity” of a protein in the context of the present invention describes the function the protein performs in its native environment. Altering the functional activity of a protein includes within its scope increasing, decreasing or otherwise altering the native activity of the protein itself. In addition, it also includes within its scope increasing or decreasing the level of expression and/or altering the intracellular distribution of the nucleic acid encoding the protein, and/or altering the intracellular distribution of the protein itself.

The terms “variant” or “derivative” in relation to Survivin polypeptide includes any substitution of, variation of, modification of, replacement of, deletion of or addition of one (or more) amino acids from or to the polypeptide sequence of Survivin. Preferably, nucleic acids encoding Survivin are understood to comprise variants or derivatives thereof.

Two additional splice variants of Survivin have been described, Survivin delta ex3 and Survivin-2B, (Mahotka et al. 1999 Cancer Research 59, 6097-6102). These isoforms may be relevant for fine-tuning Survivin activity; Survivin delta ex3, which lacks exon 3, exhibits pronounced anti-apoptotic activity whereas Survivin-2B, which contains a part of intron 2 as an additional cryptic exon, has largely lost its antiapoptotic activity, but may act as an antagonist of Survivin activity (Mahotka et al 1999). Survivin variants have also been detected in other species, indicating an evolutionarily conserved mechanism for the regulation of Survivin actions (Conway et al. 2000, Blood 95, 1435-1442; Wenzel et al. 2000; Cell Death Diff. 7, 682-683). In the context of the present invention, the term Survivin also includes within its scope splice variants such as these in as far as they possess the requisit ability to modulate neutrophil apoptosis.

The term “nucleic acid”, as used herein, refers to single stranded or double stranded DNA and RNA molecules including natural nucleic acids found in nature and/or modified, artificial nucleic acids having modified backbones or bases, as are known in the art.

An “isolated” nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide. Preferably, the term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the nucleic acids of the present invention.

“Vector” refers to any agent such as a plasmid, cosmid, virus, autonomously replicating sequence, phage, or linear single-stranded, circular single-stranded, linear double-stranded, or circular double-stranded DNA or RNA nucleotide sequence that carries exogenous DNA into a host cell or organism. The recombinant vector may be derived from any source and is capable of genomic integration or autonomous replication.

“Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5× Denhardt's solution, 10% dextran sulphate, and 20 pg/ml denatured, sheared salmon sperm DNA, followed by washing the filters in 0.1×SSC at about 65° C.

The term “myeloid cell” encompasses terminally differentiated, non-dividing (i.e. non-proliferative) cells derived from the myeloid cell lineage and includes neutrophils or polymorphonuclear neutrophils (PMNs), eosinophils and mononuclear phagocytes. The latter cells are known as monocytes when in the blood and macrophages when they have migrated into the tissues. Terminal differentiation is the normal endpoint in cellular differentiation and is usually not reversible.

“Inflammatory disorders” or “inflammatory diseases” are disorders characterised by chronic or acute inflammation. This, in turn, is characterised by elevated levels of cytokines and/or survival factors for myeloid cells. These disorders are characterised by the prolongued survival of myeloid cells including neutrophils, eosinophils and monocytes/macrophages which can be present as a mixture of one or more of these cell types. Accordingly, reference to treatment of inflammatory disorders or diseases includes treatment of the individual cell types or treatment of a mixture of different cell types. The resultant increased numbers of these inflammatory cells is associated with the disease pathology. In chronic inflammation a persistent inflammatory response causes damaging effects such as tissue damage. Chronic Inflammatory Diseases include cystic fibrosis, acute respiratory distress syndrome, chronic obstructive pulmonary disease, inflammatory bowel disease and rheumatoid arthritis. Other inflammatory diseases are known to those skilled in the art and include sepsis, Pre enclampsia, Myocardial ischemia, reperfusion injury, Psoriasis, Asthma, bronchiolitis, Crohns Disease and Ulcerative colitis.

Variants and Fragments of Survivin

In the context of the present invention the term Survivin also includes within its scope, variants, derivitives and fragments thereof, in as far as they possess the requisite ability to modulate apoptosis.

Natural variants of Survivin are likely to comprise conservative amino acid substitutions. Conservative substitutions may be defined, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Natural variants of Survivin further include splice variants such as TGN 46, 48 and 51 isoforms as described herein.

Suitable fragments of Survivin will be at least about 5, e.g. 10, 12, 15 or 20 amino acids in length. They may also be less than 100, 75 or 50 amino acids in length. They may contain one or more (e.g. 5, 10, 15, or 20) substitutions, deletions or insertions, including conserved substitutions. A fragment of Survivin used in the methods of the present invention must possess the requisite activity of being capable of modulating apoptosis.

Two additional splice variants of Survivin have been described, Survivin delta ex3 and Survivin-2B, (Mahotka et al. 1999 Cancer Research 59, 6097-6102).

Measuring Gene Expression

Levels of gene expression may be determined using a number of different techniques.

a) At the RNA Level

Gene expression can be detected at the RNA level. RNA may be extracted from cells using RNA extraction techniques including, for example, using acid phenol/guanidine isothiocyanate extraction (RNAzol B; Biogenesis), or RNeasy RNA preparation kits (Qiagen).Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U., et al., Science 242:229-237 (1988) and Lewis, R., Genetic Engineering News 10: 1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., (1994), Gynaecologic Oncology, 52: 247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B. (1989) Genomics 4:560. In the Q β Replicase technique, RNA replicase for the bacteriophage Qβ, which replicates single-stranded RNA, is used to amplify the target DNA, as described by Lizardi et al. (1988) Bio/Technology 6:1197.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., (1992) PNAS (USA) 80:392) begins with a specifically defined sequence unique to a specific target.

Primers suitable for use in various amplification techniques can be prepared according to methods known in the art.

Once the nucleic acid has been amplified, a number of techniques are available for the quantification of DNA and thus quantification of the RNA transcripts present. Methods for detection which can be employed include radioactive labels, enzyme labels, chemiluminescent labels, fluorescent labels and other suitable labels.

The detection of nucleic acids encoding Survivin can be used, in the context of the present invention, to identify early stage apoptosis in neutrophils—a decrease in Survivin transcripts is associated with the onset of apoptosis. An increase is associated with cell survival and, in particular, is an early response in GM-CSF-mediated inhibition of apoptosis in neutrophils.

b) At the Polypeptide Level

Gene expression may also be detected by measuring the Survivin polypeptide. This may be achieved by using molecules which bind to the Survivin polypeptide. Suitable molecules/agents which bind either directly or indirectly to Survivin in order to detect the presence of the protein include naturally occurring molecules such as peptides and proteins, for example antibodies, or they may be synthetic molecules.

Standard laboratory techniques such as immunoblotting can be used to detect altered levels of Survivin, as compared with untreated cells in the same cell population. An example of a suitable protocol is detailed below:

Aliquots of total protein extracts (40 μg) are run on SDS-PAGE and electroblotted overnight at 4° C. onto nitrocellulose membrane. Immunodetection involved antibodies specific for Survivin, appropriate secondary antibodies (goat, anti-rabbit or goat-anti-mouse: Bio-Rad, CA, USA) conjugated to horseradish peroxidase, and the enhanced ECL chemiluminescence detection system (Amersham, UK).

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Monitoring the Onset of Apoptosis

A number of methods are known in the art for monitoring the onset of apoptosis. These include morphological analysis, DNA ladder formation, cell cycle analysis, externalisation of membrane phospholipid phosphatidyl serine and caspase activation analysis. Cell survival may be monitored by a number of techniques including cell cycle analysis and measuring cell viability. Measurements of cell proliferation may be made using a number of techniques including a plaque assay in which adherent cells are plated out in tissue culture plates and left to grow prior to fixing and staining. The number of colonies formed reflects the amount of cell proliferation.

Modifying the Functional Activity of Survivin

The functional activity of Survivin may be modified by suitable molecules/agents which bind either directly or indirectly to Survivin, or to the nucleic acid encoding it. Agents may be naturally occurring molecules such as peptides and proteins, for example antibodies, or they may be synthetic molecules. Methods of modulating the level of expression of Survivin include, for example, using antisense techniques.

Antisense constructs, i.e. nucleic acid, preferably RNA, constructs complementary to the sense nucleic acid or mRNA, are described in detail in U.S. Pat. No. 6,100,090 (Monia et al), and Neckers et al., 1992, Crit Rev Oncog 3(1-2):175-231, the teachings of which document are specifically incorporated by reference. Suitable antisense molecules may be variants, based on these molecules, which have been chemically modified. For example, the antisense nucleic acids can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797).

Other modified oligonucleotide backbones are, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Other modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

As demonstrated herein, suitable modifying chemistries include, without limitation, the generation of phosphorothioate, methylphosphonate and locked nucleic acid derivatives as well as 2′-O-methoxy-ethoxy (2′-MOE) treatment.

Antisense modulation of Survivin expression is described, for example, in Olie et al. Cancer Research, 60, 2805-2809 and in U.S. Pat. No. 6,335,194. Since human Survivin mRNA shows extensive homology with the complmentary sequence of human EPR-1 mRNA, it is preferable to use antisense constructs which are specific to the regions unique to Survivin. An alternative strategy would be to up-regulate the expression of the “natural antisense” mRNA encoding EPR-1.

Other methods of modulating gene expression are known to those skilled in the art and include dominant negative approaches as well as introducing peptides or small molecules which inhibit gene expression or functional activity.

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391, 806-811 (1998). Other methods of PTGS are known and include, for example, introduction of a transgene or virus. Generally, in PTGS, the transcript of the silenced gene is synthesised but does not accumulate because it is rapidly degraded. Methods for PTGS, including RNAi are described, for example, in http://www.ambion.com/hottopics/rnai.

One such method involves the introduction of siRNA (small interfering RNA). Current models indicate that these 21-23 nucleotide dsRNAs can induce PTGS. Methods for designing effective siRNAs are described, for example, in http://www.ambion.com/hottopics/rnai.

In addition, changes in events immediately down-stream of Survivin, such as expression of genes whose transcription is regulated by Survivin expression, can be used as an indication that a molecule in question affects the functional activity of Survivin.

Modulator Screening Assays

Compounds having inhibitory, activating, or modulating activity can be identified using in vitro and in vivo assays for Survivin activity and/or expression, e.g., ligands, agonists, antagonists, and their homologs and mimetics.

Modulator screening may be performed by adding a putative modulator test compound to a tissue or myeloid cell sample, such as a neutrophil, and monitoring the effect of the test compound on the function and/or expression of Survivin. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds.

Myeloid cells die spontaneously in culture although with differing time courses depending on the cell type. Neutrophils in culture apoptose within 24 hours although this can be delayed to over 48 hours in the presence of survival factors. Eosinophil apoptosis is observed over 48 hours with a delay to several days in the presence of survival factors. Macrophages are generally much longer lived. Thus, the ability of a compound to modulate myeloid cell apoptosis can be assessed by monitoring the rate of apoptosis in the presence or absence of the test compound and after the withdrawal of obligate survival factors (e.g. GM-CSF, IL-8, IL-5, G-CSF or BAL) if applicable. Differences between treated and untreated cells indicates effects attributable to the test compound.

Detecting Molecules Which Interact with Survivin

Techniques such as analytical centrifugation, affinity binding studies involving chromatography or electrophoresis can be used to detect molecules which interact directly with Survivin. Those skilled in the art will appreciate that this list is by no means exhaustive. More specifically, it is possible to use Survivin as an affinity ligand to identify agents which bind to it; labeling Survivin with a detectable label and using it as a probe to detect apoptotic products in electrophoresis gels; labeling the Survivin target and using it to probe libraries of genes and/or cDNAs; labeling the Survivin target and using it to probe cDNA expression libraries to find clones synthesizing proteins which can bind to the target; performing UV-crosslinking studies to identify agents which can bind to the target; using the Survivin in gel retardation assays which would detect its ability to bind to nucleic acid encoding identified agents; performing footprinting analyses to identify the regions within a nucleic acid to which the target binds. Those skilled in the art will be aware of other suitable techniques and will appreciate that this list is not intended to be exhaustive.

Another technique that allows the identification of protein-protein interactions is immunoprecipitation. An example of a protocol for immunoprecipitation is detailed below:

For immunoprecipitation, lysates from sonicated, Triton X-100-solublized cells (60 μg protein in 100 μl PBS with protease inhibitors) are incubated for 90 min at 37° C. with 500 ng affinity-purified rabbit polyclonal antibodies specific for Survivin, followed by an addition of 10 μl packed protein A/G-agarose beads (30 min, 37° C.: Santa Cruz Biotechnology), vigorous washing of the pellet (10 min at 10000 g, 3×) in PBS, 5% SDS PAGE, and immunodetection with an Survivin-specific mAb.

Another useful technique for identifying interacting protein is the yeast-two hybrid system described, for example in Bartel et al. (eds.), The Yeast Two-Hybrid System, Oxford University Press (1997) (ISBN: 0195109384) the disclosure of which is incorporated herein by reference.

Protein interactions can also be analysed using protein arrays. These may be generated by a range of different techniques which allow proteins to be deposited on a flat surface at different densities. High density protein arrays can be generated using automated approaches similar to those described for DNA arrays (see below). Proteins interacting with Survivin may be identified by, for example, using Survivin protein to probe an expression array. Positive interactions could then be detected by the presence of, for example, a labelled antibody or by placing a tag on Survivin. The identity of the interacting protein can be determined by techniques such as mass spectrometry.

Cells

Terminally differentiated myeloid cells useful in the method of the invention may be from any source, for example from primary cultures derived from patient samples such as blood, BAL, sputum etc. or may be in vivo. Terminally differentiated cells can also be derived by differentiating cell lines including tumour cell lines of haematopoietic origin. For example tumour cell lines HL60 and MPRO (mouse) can be differentiated by retinoic acid treatment.

The invention is further described, for the purposes of illustration only, in the following examples.

### EXAMPLES

Bioinformatic Sequence Analysis Tools

DoubleTwist (www.doubletwist.com) tools were used to analyse the target sequences retrieved from Genbank. The DoubleTwist suite incorporates a number of research agents to generate computational analysis outputs using algorithms that search multiple gene, protein, and patent databases for information about query sequences. These tools access the DoubleTwist annotated databases and all published information about the query sequences. For the purpose of this study the following agents were used: Perform Comprehensive Sequence Analysis; Retrieve Assembled ESTs; Retrieve and Analyse Human Genome.

The Comprehensive Sequence Analysis agent uses the BLAST2N, BLAST2X, TBLAST2N, and BLAST2P algorithms to search the following databases: SwissProt; NR-Nuc; NR-Pro; dbEST; PDB; PAT; PATaa; HTG; Genbank's cumulative nightly nucleotide and protein database updates; and Myriad Genetics ProNet database. Additionally the Blimps and Blkprob algorithms are used to search the Blocks+ database. This agent provides information about functional protein identities and similarities, DNA identities and similarities, patented sequences, protein domains, structural identities and similarities, and genomic DNA identities and similarities.

The Assembled ESTs agent (Human) identifies matching EST clusters derived from the DoubleTwist Gene Indices. The Gene Indices are collections of assembled EST and mRNA sequences derived by, screening out non-informative sequences (such as vector and ribosomal sequences), clustering the remaining sequences, first by matching pairs for overlapping bases, then by sub-dividing into gene variants (subclusters); aligning the sequences in each cluster, and deriving a consensus sequence for each cluster and subcluster. The sequence collection is therefore checked and statistically corrected for many sequencing and cloning errors such as orientation, chimerism, and contamination. DoubleTwist's interactive data-mining tool Cluster Viewer was used to visualise the alignments.

The “Analyse Human Genome” agent also uses a proprietary DoubleTwist genome database derived from public data. Genomic sequences that are at least 15 kilobases in length are obtained from Genbank's Genomic Sequences Primate (GB PRI) division. Unfinished human genomic sequences are obtained from Genbank's High Throughput Genomic (HTG) Sequences division. The data is annotated by splitting the HTG sequences phase 0, 1, and 2 into component fragments while maintaining the GB PRI sequences intact. Sequence contamination, from vector, bacterial, yeast or mitochrondrial sequences are masked and the Repeat Masker program (http://repeatmasker.genome.washington.edu/cgi-bin/RM2_req.p1) is used to mask repetitive elements and regions of low complexity. The GrailEXP, FGENESH and Genscan algorithms are then employed to predict coding regions, introns and exons. The Halfwise algorithm is used to match predicted coding regions with models from the Pfam database. The Unigene database and the DoubleTwist Human Gene Index are further searched for DNA similarities using the BLASTN algorithm and the NR Pro database is searched, using BLASTX, for similar proteins.

The Double Twist Genomic Viewer, an interactive data mining and visualization tool was used to examine the output from the Genome Analysis agent.

The GeneTool suite from BTI (BioTools Inc) was used for sequence analysis, ClustalW (http://www.ebi.ac.uk/clustalw/) was used for creating multiple alignments. “Translate Tool” at Expasy (http://www.expasy.ch/tools/dna.html) was used to translate nucleotide sequences to protein sequences. ORF finder at the NCBI (http://www.ncbi.nlm.nih.gov/gorf/gorf.html) was used to find all open reading frames of a selectable minimum size.

### Example 1

### Gene Expression and Cluster Analysis of Neutrophil Apoptosis and Survival; Survivin is Identified by Association, as a Modulator of Apoptosis and Cell Survival in Neutrophils.

A model system for the identification of early-regulated genes in apoptosis of human primary neutrophils is described in our co-pending applications WO 01/46469 and PCT/GB01/03101.

Isolation and Culture of Primary Human Neutrophils

Whole blood (20-50 ml) is taken from normal healthy volunteers by venepuncture. Coagulation is prevented by the use of sodium citrate. A 6% dextran (mol wt 509,000; Sigma) saline solution is added in 1:4 ratio to whole blood and the erythrocytes allowed to sediment for 45 minutes at 22° C. The buffy coat is then under-layered with 5 ml Ficoll-Paque (Pharmacia LKB Biotechnology) and centrifuged (300 g, 30 min) to pellet granulocytes and erythrocytes (Boyum, 1968). The pellet is resuspended in 1 ml cell culture tested water (Sigma) for 40 sec., followed by the addition of 14 ml Hanks buffer (Sigma) and centrifuged (300 g, 10 min.). This lysis step is repeated to ensure removal of all erythrocytes. The remaining pellet is resuspended in RPMI 1640 supplemented with 10% foetal calf serum (Sigma), L-glutamine (2 mM), penicillin (100 U/ml; Sigma), streptomycin (100 μg/ml; Sigma) and amphotericin B (2.5 μg/ml; Sigma). Cell number and viability is checked using trypan blue exclusion (Boyum, (1968) Scand J Clin Lab Invest Suppl; 97:77-89).

Isolated neutrophils are maintained at a density of 2×10⁶/ml in RPMI 1640 supplemented with 10% foetal calf serum (Sigma). Further additions to the medium included L-glutamine (2 mM), penicillin (100 U/ml), streptomycin (100 μg/ml) and amphotericin B (2.5 μg/ml) (Sigma). Cells are incubated at 37° C. in a humidified CO₂ (5%) incubator. As described in by Haslett (Clinical Science 83, pp 639-648, 1992), WO 01/46469 and PCT/GB01/03101, upon culture in a serum-containing cell culture medium these neutrophils undergo spontaneous apoptosis.

Measurement of Apoptosis by DNA Fragmentation

Neutrophils are isolated from the blood as described in Example 1 and resuspended at a concentration of 2×10⁶/ml. Five hundred microlitres are pipetted into wells of a 24 well plate and incubated in the presence or absence of survival factors.

After this incubation, the neutrophils are carefully resuspended by gentle agitation and the total contents of the well are placed into an eppendorf and centrifuged @2800 rpm for 2 min @ RT in a minifuge. The cell pellet is carefully resuspended in 300 μl of ice cold hypotonic fluorochrome solution (50 μg/ml Propidium Iodide, 0.1% Sodium Citrate and 0.1% Triton X-100) and placed in a fridge for 24 h @ 4° C.

Prepared samples are analysed with a FacsCalibre flow cytometer (Becton Dickinson) using double discrimination to ensure single cell suspension with Log Forward and Side Scatter Parameter acquisition.

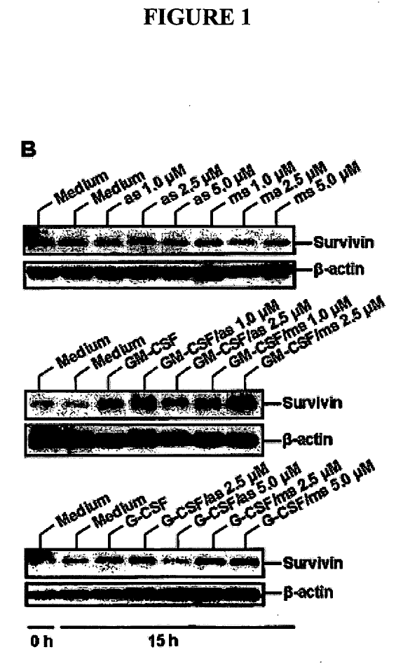

As seen in FIG. 1, healthy cells (neutrophils cultured overnight in the presence of GM-CSF) yield a single G0/G1 peak (since neutrophils are non-cycling), with few cells in the sub-G1 peak. In contrast, neutrophils cultured in the absence of GM-CSF have increased apoptosis as detected by a reduction in the amount of cells in the G0/G1 peak and enhanced number in the sub-G1 peak (indicative of DNA fragmentation).

Dose Responsiveness of the Anti-Apoptotic Effect of GM-CSF.

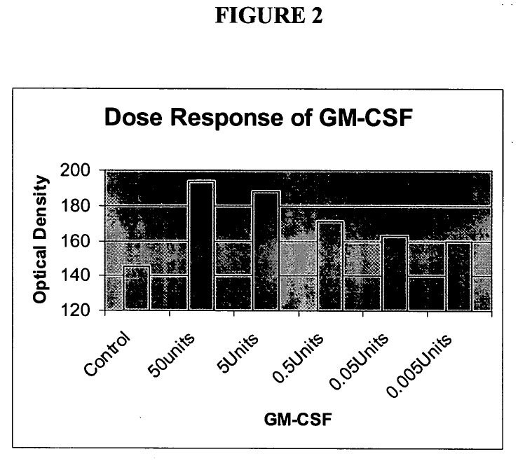

Primary human neutrophils are isolated and purified from peripheral blood of normal healthy individuals. Neutrophils are resuspended in serum containing culture medium together with various amounts of GM-CSF at a density of 2×10⁶/ml, with 100 μl plated into a 96 well plate and cultured for 18 h at 37° C. After this time 10 μl of MTT (5 mg/ml) is added to the cultures and incubated for a further 4 h at 37° C. before solubilisation of the purple coloured formazan with acidic isopropanol. Optical densities are read at 570 nm using a plate reader. FIG. 2 shows there is a direct correlation between survival and concentrations of GM-CSF added to the culture medium.

For subsequent experiments neutrophils are resuspended in serum containing culture medium containing 5 U/ml of GM-CSF.

Fungal Metabolite Gliotoxin Blocks GM-CSF Inhibition of Neutrophil Apoptosis.

This describes the identification of an inhibitor for the GM-CSF mediated inhibition of neutrophil apoptosis. The use of this inhibitor allows us to focus in on the specific biochemical events mediating the GM-CSF survival events. In turn one is able to remove some of the noise associated GM-CSF treatment.

Primary human neutrophils are isolated and purified from peripheral blood of normal healthy individuals. Neutrophils are resuspended in serum containing culture medium containing 5 U/ml of GM-CSF at a concentration of 2×10⁶/ml. Also added to the culture mix is either 0.1 μg/ml of the fungal metabolite Gliotoxin or its inactive analogue bis-Dethio-bis (Methylthio) Methyl Gliotoxin, with 100 μl/well plated into a 96 well plate and culture at 37° C. commenced. After the indicated time, 10 μl of MTT (5 mg/ml) are added to the cultures and incubated for a further 4 h at 37° C. before solubilisation of the purple coloured formazan with acidic isopropanol. Optical densities are read at 570 nm using a plate reader.

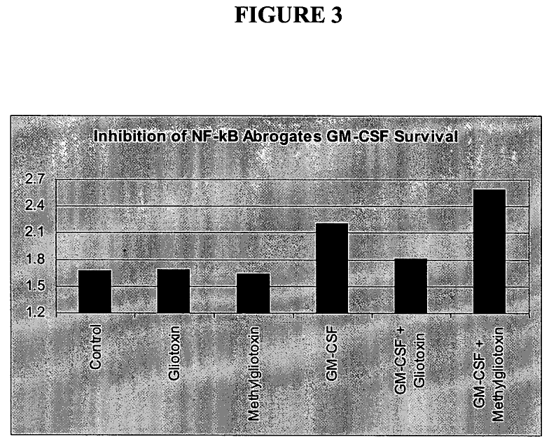

FIG. 3 demonstrates that gliotoxin effectively blocks the GM-CSF inhibition of neutrophil apoptosis. This blocking effect is not seen when the inactive analogue of gliotoxin, methylgliotoxin is added with GM-CSF. No increased neutrophil apoptosis is seen with the addition of gliotoxin alone to isolated neutrophils demonstrating that the effect is specific to and limited to a reversal of the protective effects of GM-CSF.

Commercial microarrays are used to measure global gene expression associated with neutrophil apoptosis, GM-CSF inhibition of neutrophil apoptosis, and the inhibition of this effect using the fungal metabolite Gliotoxin. In control experiments, an inactive analogue of Gliotoxin, Methyl Gliotoxin is used. Analysis of such microarray results identifies genes whose expression pattern changes (either up-regulation or down-regulation) in an association with a measurable apoptotic phenotype.

Total RNA Isolation

Primary human neutrophils are isolated and purified from peripheral blood of normal healthy individuals using standard techniques. Neutrophils are resuspended in serum containing culture medium together with GM-CSF (50 U) at a concentration of 2×10⁶/ml, and cultured for 0 h (control), 2 h, 4 h and 6 h at 37° C. Total RNA is then prepared from both groups using acid phenol/guanidine isothiocyanate extraction (RNAzol B; Biogenesis), or RNeasy RNA preparation kits (Qiagen). Any contaminating genomic DNA is removed by DNase treatment (DNase I, Gibco-BRL).

RNA is also prepared from neutrophil cells following treatment (for the time indicated in hours) with GM-CSF (50 units/ml), Gliotoxin (10 μM) or MethylGliotoxin (10 μM). RNA is also prepared from neutrophils that have not been exposed to drug (i.e. as an untreated control). RNA is prepared from these cells using two sequential extractions with RNAzol B.

Measurement of Global Gene Expression by ‘Microarray’

The process of microarraying can be used to profile gene expression of thousands of genes simultaneously. The microarray process is described both for the use of Human LifeGrid™ microarray filters and can be separated into three parts: the filter, the hybridisation of radiolabelled cDNA probe, and the detection and quantitation of the microarray results.

The Microarray Filter

This example describes the use of the Human LifeGrid™ microarray filters obtained from Incyte Genomics (USA). These filters contain cDNA probes representing approximately 8,400 human mRNAs.

Hybridisation of Radiolabelled cDNA Probes.

This example describes the synthesis of a radiolabelled cDNA from total cellular mRNA. The labeled cDNA is used to ‘probe’ DNA fragments, which have been immobilised on to a filter membrane, by complementary hybridisation.

Methodology is as described by manufacturer, for Human LifeGrid™ arrays. Essentially, total cellular RNA (1 μg to 20 μg) or polyA+mRNA (100 ng to 5 μg) is incubated with an oligo (dT) primer. Primed RNA is reverse transcribed to first strand cDNA in a reaction containing M-MLV reverse transcriptase (RT; alternatively Superscript II is used (Life Sciences)), RT buffer, dNTPs and [α-³³P] dCTP (2000-4000 Ci/mmol) at 42° C. for 1 to 5 hours. Unincorporated nucleotides are removed using spin-columns and the labeled probe stored at −80° C. until required.

Labeled probes may also be generated from cDNA, genomic DNA or PCR products. In each case a random primed labeling procedure can be used, for example the Ready-Prime Labeling kit (APBiotech), applied as per manufacturers instructions.

Radiolabelled cDNA probe is hybridised to DNA fragments immobilised onto a membrane (typically a nylon or nitrocellulase filter).

Methodology is as described by manufacturer, for Human LifeGrid™ arrays. Essentially, membrane filters are pre-hybridised in hybridisation buffer (5 to 20 ml) at 42° C. for 2 to 16 h using a hybridisation oven (Hybaid). Following pre-hybridisation, the labeled cDNA probe is added to fresh hybridisation buffer (5 to 20 ml) and hybridised at 42° C. for 14 to 16 h. Following hybridisation, the hybridisation mix is removed and the filters washed with 2×SSC buffer at RT for 5 min., twice with 2×SSC, 1% SDS buffer at 68° C. for 30 min. and twice with 0.6×SSC, 1% SDS buffer at 68° C. for 30 min.

Detection and Quantitation of the Microarray Results.

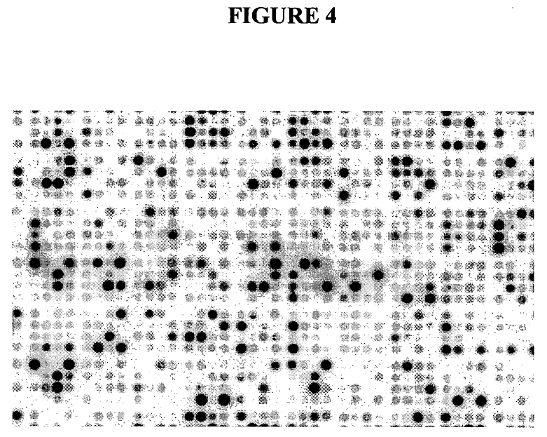

This example describes the use of a STORM Phosphoimager to quantitatively image positive signals across the filter arrays. Hybridised filters are wrapped in plastic wrap (Saran) and exposed to a Low-Energy Phosphoimaging screen (Molecular Dynamics). The screen is then placed on the phosphoimager and the gel image captured by scanning at a resolution of 50 microns (See FIG. 4).

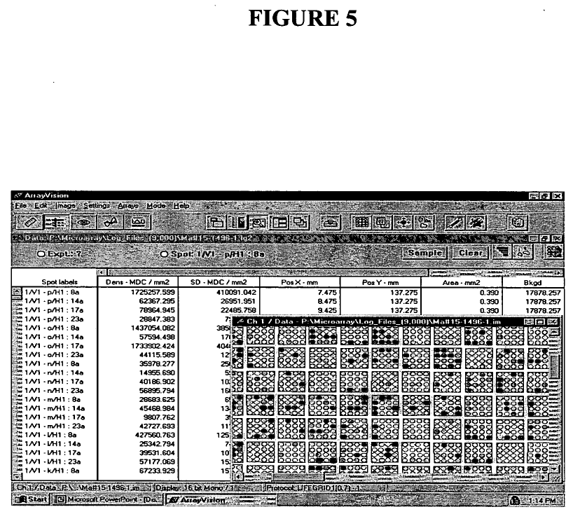

The captured image file is then analysed using software such as Array Vision (Imaging Research Inc.; See FIG. 5). In this example we implement analysis with ArrayVision v5. 1. This program contains facilities for spot detection and quantification, and background detection and quantification. This data is then exported to a text file for further analysis. A variety of data fields are exported from the ArrayVision analysis, including; Spot Label, Position, Density, Background, and particularly, Background subtracted density (sDens) and signal/noise ratio (S/N). In this example, the exported text file is up-loaded to an SQL-7.0 database, to populate a table containing array data from all experiments. As the data is imported to the database, a Normalisation factor is calculated and the sDENs values modified accordingly. This Normalised data is stored in a newly created column within the table. The Normalisation factor facilitates accurate comparison between datasets. A number of different calculations may be used. A normalization factor may be derived from Linear Regression calculated by reference to housekeeping genes. Alternatively, the Global Mean is calculated as the average of the sDens values across all of the arrays to be compared and a normalisation factor is then derived by division of the overall spot density with the Global Mean value. Spot density values (individual sDens) are then corrected by multiplying across all values with the normalisation factor. In a similar approach a Global Geometric Mean normalization factor may be calculated and used to adjust the dataset. The data from multiple hybridisation experiments can then be stored in a suitable format, for example in an Access or SQL 7.0 database.

Comparison between arrays generates an output file containing the gene identifier and the fold-change in expression relative to the reference dataset. Fold change, (Tx vs Ty), is calculated by dividing the normalised spot density values of Tx with Ty. In this example, multiple time-course experiments are prepared and fold-change values calculated with reference to the T0 time point.

The fold change data derived from comparison of multiple hybridisation experiments can be analysed using a variety of approaches, including hierarchical clustering, (supervised or unsupervised), k-means clustering or self-organising maps. Software enabling these analyses includes the Cluster and Treeview software (M. Eisen, Stanford Uni, USA), J-Express (European Bioinformatics Institute), GeneMaths (Applied Maths, Belgium) or GeneSpring (Silicon Genetics, USA). In this example hierarchical clustering is implemented using the GeneMaths software. Trees are generated using the UPGMA algorithm with distance calculated using the Pearson similarity metric. Alternatively Euclidean distance metrics are used.

Simplification of Fold-Change Data

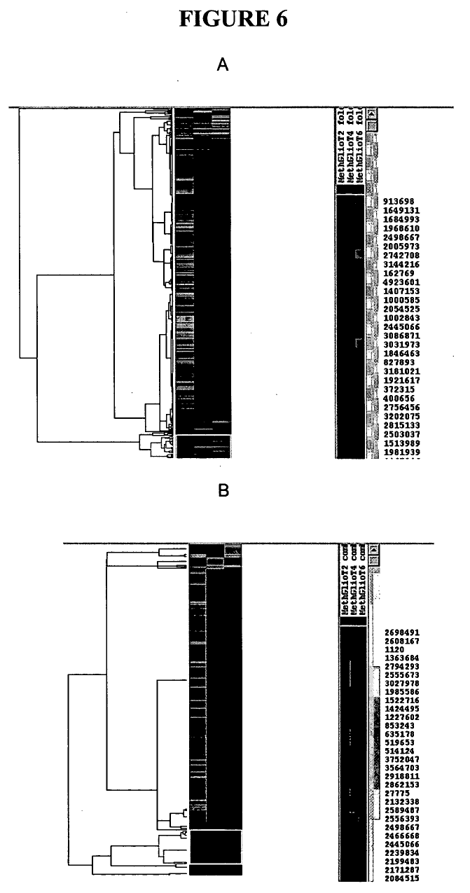

Following cluster analysis, fold-change data can be difficult to interpret owing to either a very large dataset and/or a wide range in fold change values. The visualization and interpretation of these datasets may be simplified using codes or combined codes. In this example, each unique gene is represented by at least two identical cDNAs on the array. The fold change value is calculated as described, then for each spot, a value above 5-fold change is accorded a code of 2, a fold-change value of less then 5 but greater then 2 is accorded a code of 1 and a fold-change value of less then 2 is accorded a code value of 0. A combined code is then derived by adding the code values for each identical cDNA on the array. The use of combined codes can greatly simplify the Cluster analysis and the subsequent visualisation (See FIG. 6).

Comparison of coordinate patterns of gene expression, by bioinformatic data analysis, using this model system, allows the identification of cell pathways and processes associated with apoptosis and survival.

In any given experiment or time course, ‘differentially regulated’ genes (combined code greater than or equal to 2) are identified and clustered by either normalised sDens (level of expression) or by fold change values. Candidate genes, associated with apoptosis and survival, are those that are reproducibly differentially regulated in multiple experiments or time courses and are additionally ‘reciprocally regulated’ in conditions that permit apoptosis versus survival, respectively.

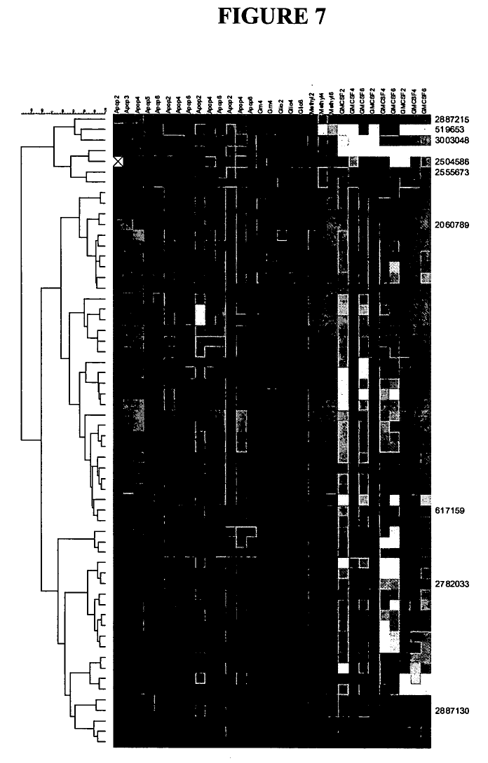

FIG. 7 shows the visual representation of a clustered selection of candidate neutrophil apoptosis/survival-associated genes identified from LifeGrid filters. Each row represents the differential regulation of an individual gene. The Fold Change colour scale is shown.

Experiments measuring neutrophil apoptosis, GM-CSF inhibition of apoptosis and Gliotoxin blockage of GM-CSF inhibition of apoptosis were as follows:

Neutrophil Apoptosis

Four representative neutrophil apoptosis time course experiments are represented (Apop), with RNA samples isolated at 2 h (Apop2), 3 h (Apop3), 4 h (Apop4), 5 h (Apop5) and 6 h (Apop6) post-isolation of neutrophils. Fold change values are expressed relative to zero hour control samples.

Inhibition of Neutrophil Apoptosis by Treatment with GM-CSF

Three representative GM-CSF time course experiments are represented (GM-CSF), with RNA samples isolated at 2 h (GMCSF2), 4 h (GMCSF4) and 6 h (GMCSF6) post-treatment with GM-CSF. Fold change values are expressed relative to zero hour control samples.

Blockage of GM-CSF-Mediated Inhibition of Neutrophil Apoptosis by Treatment with Gliotoxin

Three representative Gliotoxin time course/experiments are represented. In one, GM-CSF is added in the presence of Gliotoxin (Glio) or an inactive analogue Methyl Gliotoxin (Methyl), with RNA samples isolated at 2 h (Glio2 and Methyl2), 4 h (Glio4 and Methyl4) and 6 h (Glio6 and Methyl6) post-treatment with GM-CSF. Fold change values are expressed relative to zero hour control samples. In the remaining two experiments (GM 4) RNA samples are isolated at 4 h post-treatment with GM-CSF, and fold change values are expressed relative to Methyl Gliotoxin control samples.

Each experimental RNA sample, profiled by microarray, represents the pool of multiple experiments carried out on neutrophils isolated from individual human donors. The number of donor samples used for each experiment/time course is summarised in Table 1.

| Experiment | Donors |
|---|---|
| Apop 2, 3, 4, 5, 6 | n = 7 |
| Apop 2, 4, 6 | n = 17 |
| Apop 2, 4, 6 | N = 8 |
| Apop 2, 4, 6 | n = 8 |
| GM4 | n = 7 |
| GM4 | n = 7 |
| Glio 2, 4, 6 | n = 3 |
| Methyl 2, 4, 6 | n = 3 |
| GMCSF 2, 4, 6 | n = 9 |
| GMCSF 2, 4, 6 | n = 12 |
| GMCSF 2, 4, 6 | n = 11 |

Average fold change values (from two spots on the array filters) are clustered with GeneMaths, using a Pearson correlation and Ward clustering algorithm.

Candidate genes represented in this selection share similar overall expression characteristics, that of an ‘apoptosis/survival cluster’. Candidate genes tend to be down-regulated (dark) in multiple experiments and time courses for apoptosis (Apop, GM and Glio; see legend) and up-regulated (light) in experiments and time courses for survival (Methyl and GMCSF; see legend).

One of the differentially expressed genes associated with apoptosis and survival is identified as Survivin.

### Example 2

Survivin mRNA is Increased in GM-CSF-Induced Neutrophil Survival, and this Increased Expression is Blocked by Gliotoxin

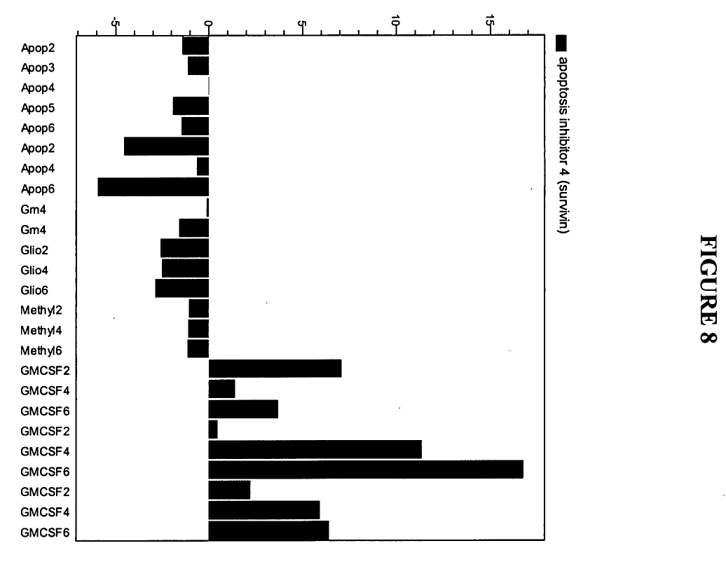

FIG. 8 shows the relative amounts of Survivin transcripts isolated from neutrophils treated according to Example 1. Experimental conditions and cluster analysis of average fold change comparisons are as described in Example 1.

Expression of Survivin is up-regulated in multiple experiments between 2 and 6 h following addition of GM-CSF. Up-regulated genes may represent potential survival factor genes, which block or delay the apoptosis in neutrophils. Increased expression of Survivin, following GM-CSF treatment, is blocked by the fungal inhibitor gliotoxin (Glio and GM; see legend).

### Example 3

Cluster Analysis and Correlation and Association of Survivin with Survival in Myeloid Cells, the Neutrophil.

In our co-pending applications WO 01/46469 and PCT/GB01/03101, we have established that gene function can be predicted by correlation to known genes that have a similar pattern of gene expression across multiple experiments. The use of bioinformatics cluster analysis to identify novel pathways and gene function is also described, for example, by Zhao et al. PNAS 98(10): 5631-5636, (2001); Heyer L J et al. Genome Res. 9(11):1106-15, (1999); Iyer V R et al. Science 283(5398):83-7, (1999); and in Gene Expr 7(4-6):387-400 (1999).

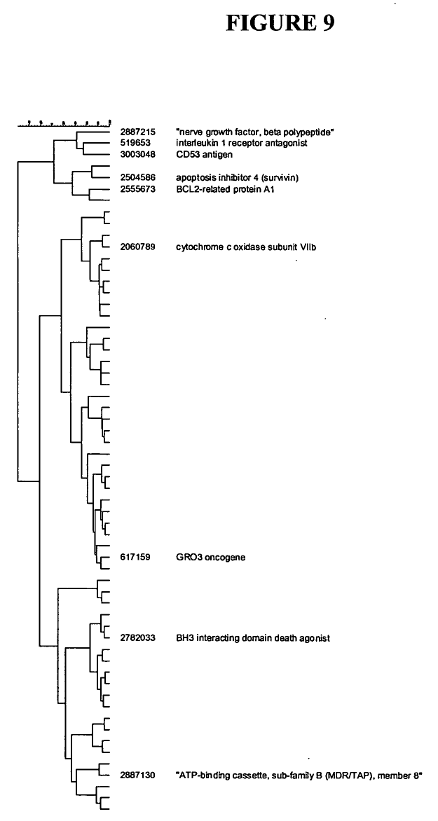

FIG. 9 shows a dendrogram representation of the association of candidate genes from the cluster analysis illustrated in FIG. 7 (performed using the method detailed in Example 1) of Survivin expression compared to other known genes that have a similar pattern of gene expression across multiple experiments. Amongst these are cytochrome c oxidase subunit VIIb (2060789), BH3 interacting domain death agonist (2782033), BCL2-related protein A1 (2555673), CD53 antigen (3003048), interleukin 1 receptor antagonist (519653), ATP-binding cassette, sub-family B (MDR/TAP), member (2887130), GRO3 oncogene (617159), stratifin (2028680) and nerve growth factor, beta polypeptide (2887215). All of these genes are known to be involved in apoptosis and survival. Several, including cytochrome c oxidase, CD53 and interleukin 1 receptor antagonist are also associated with Redox regulation.

Cytochrome c oxidase (COX), the terminal component of the respiratory chain complex of most aerobic organisms, is composed of 13 subunits in mammals. Mitochondrial release of cytochrome c is one of the principle stepps initiating the execution of apoptosis. Mitochondrial antisense RNA for cytochrome C oxidase can induce morphologic changes and cell death in human hematopoietic cell lines (Blood 1997 Dec. 1;90(11):4567-77). Apoptosis and ROS detoxification enzymes correlate with cytochrome c oxidase deficiency in mitochondrial encephalomyopathies (Mol Cell Neurosci 2001 April;17(4):696-705).

BH3 interacting domain death agonist, otherwise known as BID, is activated by the pro-apoptotic cascade. This causes BID to oligomerize BAK or BAX into pores that result in the release of cytochrome c. (for review see Cell Death Differ 2000 December;7(12):1166-73).

BCL2-related protein A1, otherwise known as Bfl-1 was first isolated by Lin et al. (1993) as a novel mouse cDNA sequence, designated BCL2-related protein A1 (Bcl2a1) and was identified as a member of the Bcl-2 family of apoptosis regulators by the predicted protein sequence. An anti-apoptotic role of Bfl-1 is described in staurosporine-treated B-lymphoblastic cells (Int J Hematol 2000 December;72(4):484-90).

CD53 is an N-glycosylated pan-leukocyte antigen of 35,000 to 42,000 MW. Increased expression of CD53 has been described on apoptotic human neutrophils (J Leukoc Biol 2000 March;67(3):369-73). Voehringer D W et al, described CD53 associated with resistance to ionising radiation, using microarray experiments. Expression of CD53 can lead to the increase of total cellular glutathione, which is the principle intracellular antioxidant and has been shown to inhibit many forms of apoptosis (Proc Natl Acad Sci USA 2000 Mar. 14;97(6):2680-5).

The Inter Leukin 1 receptor antagonist (IL1RN) is a protein that binds to IL1 receptors and inhibits the binding of IL1-alpha and IL1-beta. Overexpression of interleukin-1 receptor antagonist provides cardioprotection against ischemia-reperfusion injury associated with reduction in apoptosis (Circulation 2001 Sep. 18;104(12 Suppl 1):I308-I3). Hypoxia induces the expression and release of interleukin 1 receptor antagonist in mitogen-activated mononuclear cells (Cytokine 2001 Mar. 21;13(6):334-41). Overexpression of IL-1ra gene up-regulates interleukin-1beta converting enzyme (ICE) gene expression: possible mechanism underlying IL-1beta-resistance of cancer cells (Br J Cancer 1999 September;81(2):277-86).

ATP-binding cassette, sub-family B (MDR/TAP) is homologous to MDR1 (multiple drug resistance). Increased expression and amplification of MDR1 sequences were also found in multidrug-resistant sublines of human leukemia and ovarian carcinoma cells. Overexpression of MDR1 appears to be a consistent feature of mammalian cells displaying resistance to multiple anticancer drugs and has been postulated to mediate resistance.

GRO3 oncogene: The GRO gene, a CXC chemokine otherwise known as macrophage inflammatory protein 1 beta (MIP-1B), was initially identified by Anisowicz et al. (1987) by its constitutive overexpression in spontaneously transformed Chinese hamster fibroblasts. Neutrophils have been shown regulate their own apoptosis via preservation of CXC receptors. Gro-alpha and IL-8 (CXC chemokines) suppress neutrophil apoptosis (Neu J Surg Res 2000 May 1;90(1):32-8).

Stratifin is one of the 14-3-3 family of proteins which mediate signal transduction by binding to phosphoserine-containing proteins. The 14-3-3 dimer binds tightly to single molecules containing tandem repeats of phosphoserine motifs, implicating bidentate association as a signaling mechanism with molecules such as Raf, Cbl and the pro-apoptotic molecule BAD (Cell 91: 961-971, 1997). Stratifin, is strongly induced by gamma irradiation and other DNA-damaging agents. The induction of 14-3-3-sigma is mediated by a p53-responsive element located 1.8 kb upstream of its transcription start site. Exogenous introduction of 14-3-3-sigma into cycling cells results in a G2 arrest (Molec. Cell 1: 3-11, 1997).

Nerve growth factor, beta polypeptide: Nerve growth factor is a well-characterised cytokine survival factor. NGF withdrawal induces apoptosis in a range of cells in-vitro and in-vivo. Nerve growth factor suppresses apoptosis of murine neutrophils (Biochem Biophys Res Commun 1992 Jul. 31;186(2):1050-6).

The close association of Survivin gene expression, across multiple reciprocal experiments, with a significant number of know apoptosis and survival genes identifies a function for Survivin in neutrophil and cellular apoptosis and survival.

### Example 4

Expression of Multiple Survivin Isoforms in Neutrophil and HL60 Cells

Neutrophils were purified as described in Example 1. RNA was prepared from neutrophils treated with GMCSF. Non-quantitative polymerase chain reaction amplifications were prepared according to standard conditions using primers

| | |
|---|---|
| F1 | |
| 5′-ATGGGTGCCCCGACGTTGC-3′ | (Predicted Tm: 59° C.) |
| R1 | |
| 5′-TCAATCCATGGCAGCCAGCTG-3′ | (Predicted Tm: 57° C.) |

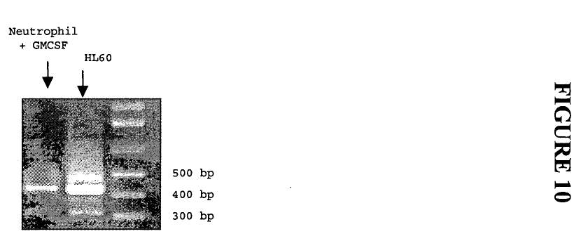

These primers will amplify fragments of 429 bp (Survivin), 498 bp (Survivin-2B) and 311 bp (Survivin-□ex3). The results of this experiment are presented in FIG. 10, and illustrate that all three isoforms are expressed in neutrophils treated with GMCSF. A control sample derived from HL60 cells is also shown.

### Example 5

Survivin Expression in Neutrophil Differentiation.

Survivin expression increases upon differentiation of myeloid cells to mature neutrophils.

Nitroblue Tetrazolium (NBT) Reduction Test

HL60 cells are plated in 75 cm² flasks at a concentration of 0.5×10⁶/ml in RPMI+10% FCS (20 ml/flask) and incubated for the indicated period of time with 10 μM Retinoic acid after which time 1.5×10⁶ cells trypan blue negative cells are resuspended in 1 ml of RPMI medium and stimulated with 50 ng/ml Phorbal Myristate Acetate (PMA, Sigma) for 2 minutes. Nitroblue Tetrazolium salt is added to a final concentration of 50 μg/ml. Following incubation for 15 minutes at 37° C. the samples are placed on ice to terminate the reaction. Cells are then centrifuged at 300×g for 5 minutes and the supernatant removed. Cells are washed once in PBS and resuspended in 1 ml PBS. Cells are then cytocentrifuged onto glass slides using a cytospin (Shandon II). Slides are allowed to air dry and cells are then fixed in methanol (Rapi Diff Kit; Diachem Int, UK). A counter stain is applied by immersing the slides in an Eosin stain (Solution B; Rapi Diff Kit; Diachem Int, UK) for 10 minutes. Excess stain is removed by gentle washing with water. The slide is then air-dried and a cover slip applied. Positive and negative cells are enumerated at 40× magnification. Slides are assessed blind at 3 different fields and the mean calculated. NBT positive cells are determined as those that contained blue intracellular deposits. NBT negative cells are stained pink and are free of blue particles.

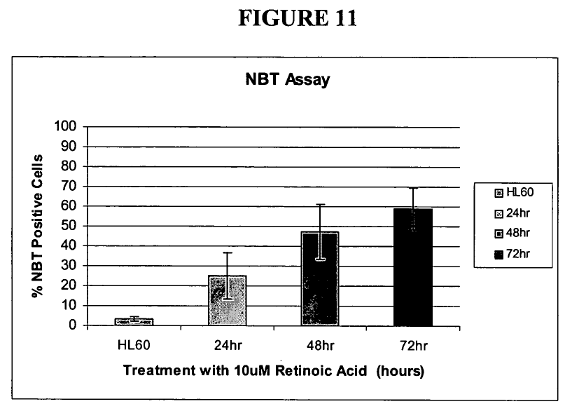

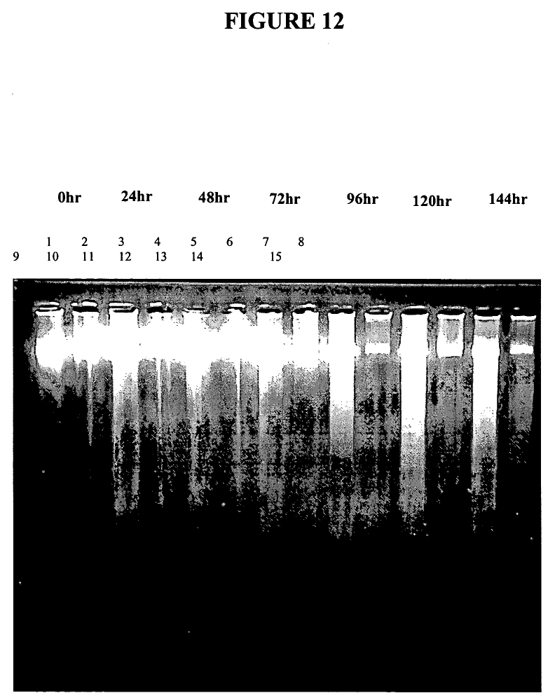

FIGS. 11 and 12 show time courses of neutrophil differentiation and apoptosis, respectively. Upon treatment of HL60 cells with retinoic acid, HL60 cells arrest their cell cycle and differentiate into neutrophils across a 5-day time course. Markers of differentiated neutrophils are increasingly detected at day 2 and day 3, as measured by reduction of NBT (see FIG. 11 and also Martin S J et al, Clin. Exp. Immunol. (1990)). Apoptosis begins to occur around day 4 (96 h) as shown in FIG. 12.

At the time points indicated RNA samples are isolated by lysing cells and adding RNAzol (5×10⁶ cells/ml RNAzol), purifying RNA as described previously and analysed by microarray using Incyte LifeGrid filters as described previously.

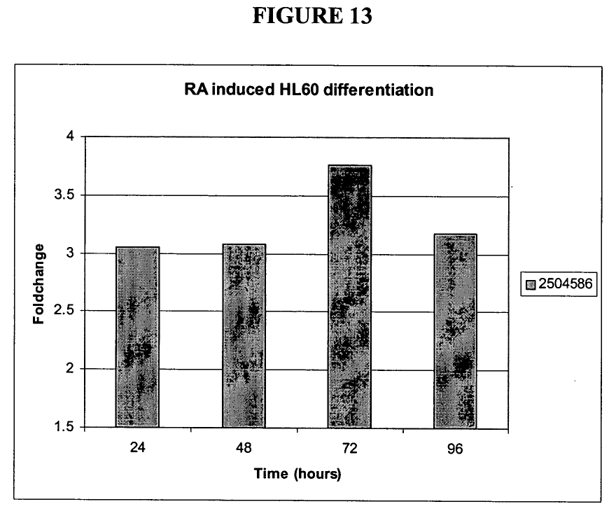

FIG. 13 shows Survivin gene expression fold change in retinoic acid treated HL60 cells.

Expression of Survivin increases throughout Day 1, 2 and 3 correlating with cell cycle arrest and neutrophil differentiation, and decreases on Day 4 correlating at the time when apoptosis is occurring.

These results demonstrate that Survivin expression is associated with the process of neutrophil differentiation and apoptosis following treatment with retinoic acid.

### Example 6

Survivin is Expressed at Markedly Increased Levels in Neutrophils Exposed to Survival Factors in vitro and Inflammatory Cells, such as Neutrophils Isolated from Patients with the Chronic Inflammatory Disease, Cystic Fibrosis.

Cells

Peripheral blood neutrophils are purified from healthy normal individuals or patients suffering from cystic fibrosis by Ficoll-Hypaque centrifugation (Dibbert et al. (1999) Proc. Natl. Acad. Sci. USA 96, 13330-13335; Yousefi et al. (1994) Proc. Natl. Acad. Sci. USA 91, 10868-10872). The resulting cell populations contain more than 95% neutrophils.

Cell Cultures

Neutrophils are cultured at 1×10⁶ per ml in complete culture medium. GM-CSF (50 ng/ml; kind gift of T. Hartung, University of Konstanz, Konstanz, Germany) or G-CSF (25 ng/ml; PeproTech, BioConcept, Allschwil, Switzerland) are added. The duration of GM-CSF or G-CSF stimulation is indicated in each experiment.

Real-Time PCR

Neutrophils (1×10⁷) are washed with PBS and the RNA is isolated according to the RNeasy protocol (Qiagen AG, Basel, Switzerland), which includes DNase digestion. RNA is reverse transcribed with the first strand cDNA synthesis kit (Amersham Pharmacia Biotech) by using random hexanucleotides according to the manufacturer's instructions. Real-time monitoring of PCR amplification of survivin cDNA is performed with a 1/100 dilution of neutrophil cDNA as previously described (Olie et al. (2000) Cancer Research 60, 2805-2809). Relative quantification of survivin expression is performed as described (Olie et al. (2000) Cancer Research 60, 2805-2809) utilizing rRNA as an internal standard.

Statistical Analysis

Statistical analysis is performed by using Student's t test. A p value of <0.05 is considered statistically significant. Mean levels are presented together with standard errors of the mean (S.E.M.). In the antisense oligonucleotide experiments, antisense and mismatch survivin—treated cells are compared at each indicated concentrations.

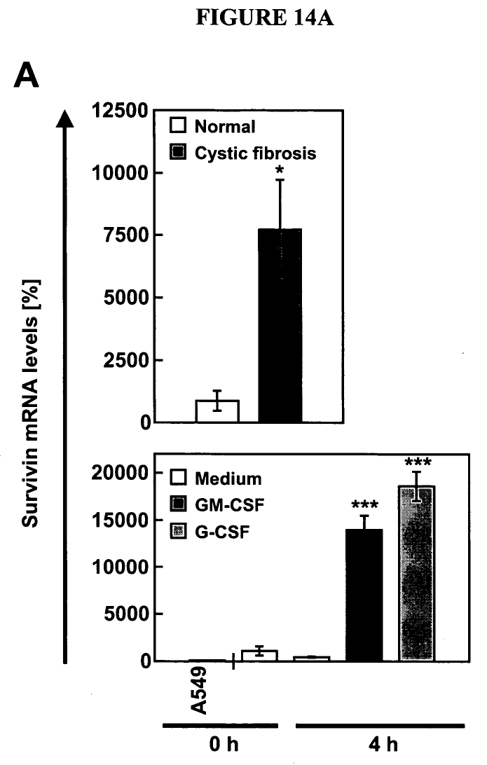

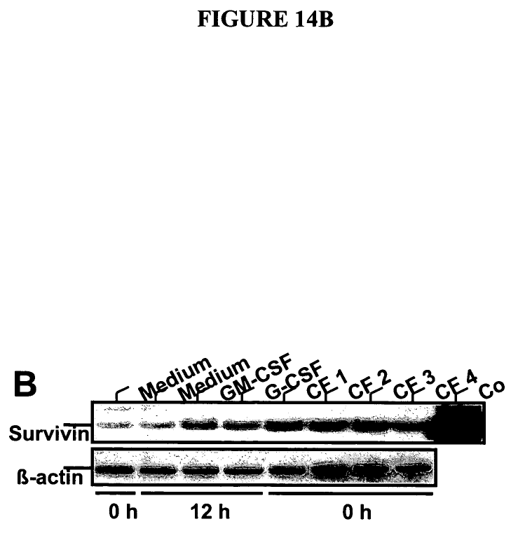

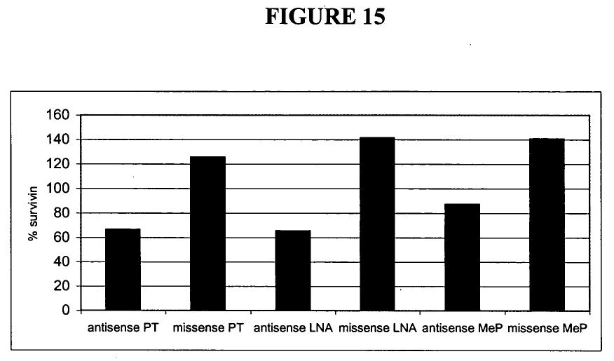

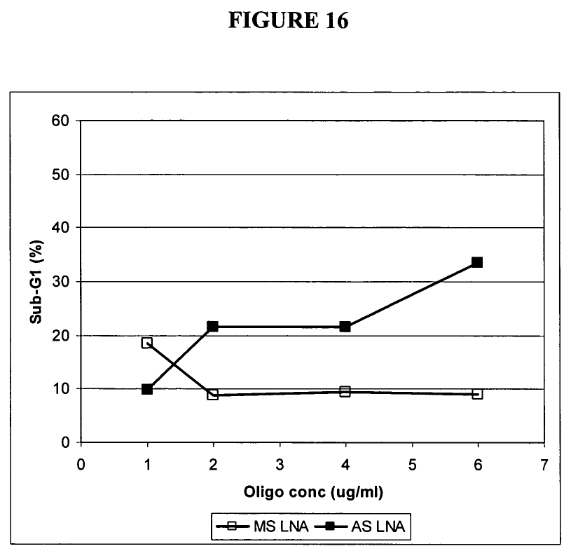

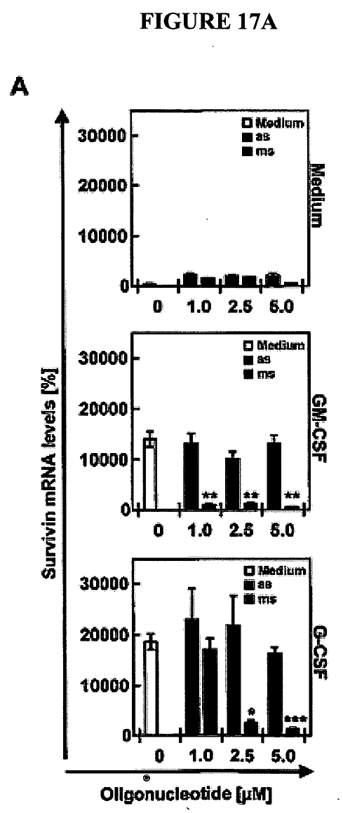

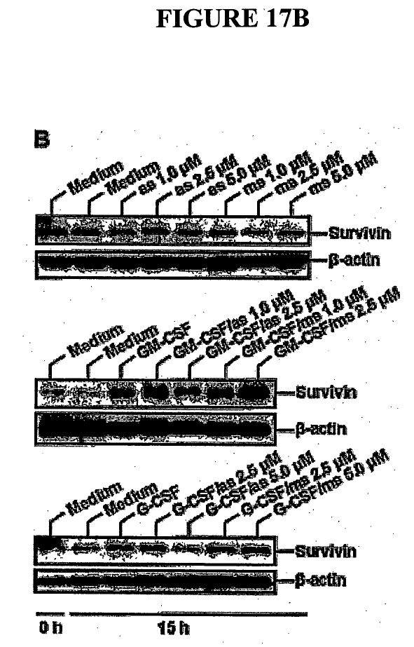

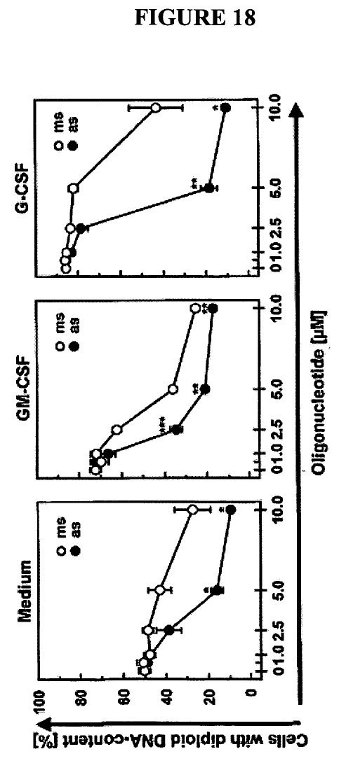

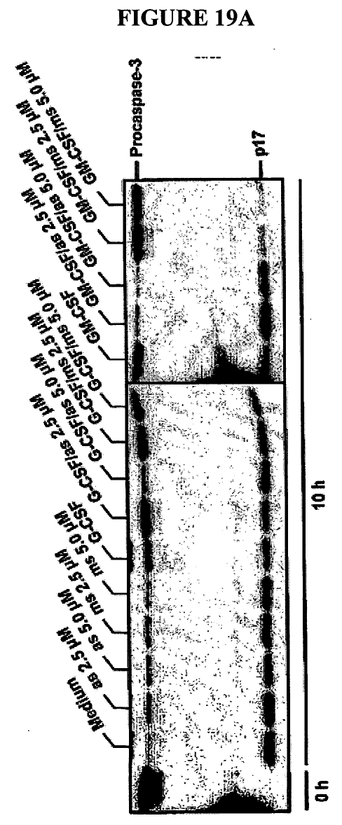

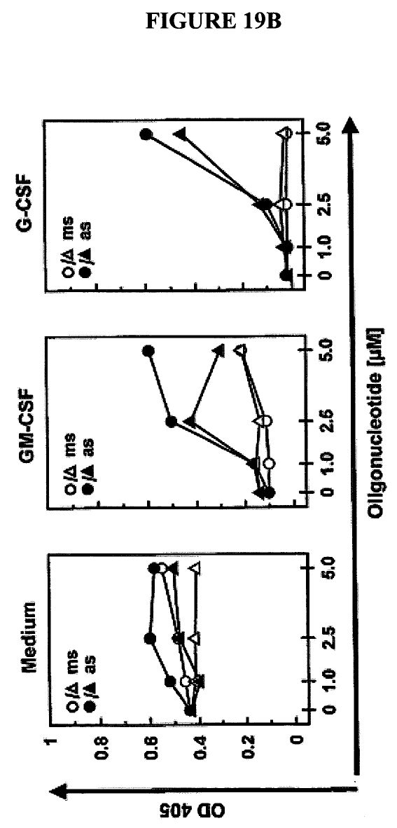

FIG. 14A shows real-time PCR quantification of survivin mRNA. The lung cancer cell line A549 serves as a standard (=100%). Cystic fibrosis neutrophils contain increased amounts of survivin mRNA compared to normal neutrophils (upper panel). The increase in survivin mRNA in cystic fibrosis neutrophils is mimicked by addition of the inflammatory cytokines GM-CSF and G-CSF to normal control neutrophils in vitro (lower panel).

Gel Electrophoresis and Immunoblotting

Neutrophils (5×10⁶) are washed with PBS, lysed with RIPA-buffer (0.5% sodium deoxycholate, 1% Nonidet P-40, 0.1% SDS in PBS) supplemented with a protease inhibitor-cocktail (Sigma) with frequent vortexing on ice for 40 min, including two freeze/thaw cycles using a methanol/dry ice bath. After a 10-min centrifugation to remove insoluble particles, equal amounts of the cell lysates are loaded on NuPage-Gels (Invitrogen Corp., Groningen, Netherlands). Separated proteins are electrotransferred onto polyvinylidene difluoride (PVDF) membranes (Immobilion-P, Millipore, Bedford, Mass.). The filters are incubated overnight with a polyclonal anti-survivin antibody (1/1000; R&D Systems, Abingdon, UK) at 4° C. in TBS/0.1% Tween-20/3% non-fat dry milk. Purified full-length recombinant human survivin (2 μl) is provided by R&D Systems as positive control for anti-survivin immunoblotting. For loading controls, stripped filters are incubated with a monoclonal anti-β-actin antibody (1/10,000; Sigma). Filters are washed in TBS/0.1% Tween-20 for 30 min and incubated with the appropriate HRP-conjugated secondary antibody (Amersham Pharmacia Biotech, Dübendorf, Switzerland) in TBS/0.1% Tween-20/5% non-fat dry milk for 1 h. Filters are developed by an ECL-technique (ECL-Kit, Amersham Pharmacia Biotech) according to the manufacturer's instructions.

FIG. 14B shows levels of survivin in neutrophils are increased by culturing normal control neutrophils for 12 h in the presence of GM-CSF and G-CSF. In addition, freshly isolated neutrophils of patients suffering from cystic fibrosis (CF, n=4) show strongly increased amounts of survivin compared to normal control individuals (first lane). Purified recombinant survivin serves as positive control (Co). β-actin immunoblotting is performed to demonstrate equal loading.

Bronchoalveolar lavage (BAL) fluid is obtained from patients with chronic inflammatory disease, including cystic fibrosis, chronic obstructive pulmonary disease or healthy controls. RNA is prepared from cells contained in the lavage according to standard protocols (example 1). Polymerase chain reaction amplification for Survivin cDNA is prepared as described in Example 4, using primers F1 and R1.

Results indicate Survivin is expressed in cells of BAL from patients with inflammatory disease.

### Example 7

Survivin Antisense Oligonucleotides Inhibit the Neutrophil Survival Effect of Bronchoalveolar Lavage from Chronic Inflammatory Disease.

Neutrophils are purified as normal and resuspended in culture medium (RPMI+10% FCS) to give a final conc. of 2×10⁶/ml. Five hundred microlitres are added to each well of a 24 well plate, to give a final concentration of 1×10⁶/well.

Suvivin antisense oligonucleotide sequence #4003: Olie RA et al. A novel antisense oligonucleotide targeting Survivin expression induces apoptosis and sensitizes lung cancer cells to chemotherapy. Cancer Res 2000 Jun. 1;60(11):2805-9. These are chosen to block expression of Survivin and the two splice variants, Survivin-2B and Survivin-Δex3.

Either antisense or missense oligonucleotides are added directly to each well over a range of concentrations between 0 μM to 10 μM.

Sequence for antisense oligonucleotide is:
5′-CCCAgCCTTCCAgCTCCTTg-3′
Sequence for missense (mismatch) oligonucleotide
is:
5′-CCTAgCCTTCCAggTCCTAg-3′

Cells are then incubated for time intervals between 15 mins to 8 hours. Bronchoalveolar lavage (BAL) fluid, obtained from patients with chronic inflammation, including cystic fibrosis, chronic obstructive pulmonary disease or healthy controls, is then added to each well. As a positive control for increased survival, neutrophils are treated with GM-CSF (0 to 20 units).

Neutrophil apoptosis is measured as described in Example 1.

Addition of antisense, but not missense Survivin oligonucleotides inhibits the survival of neutrophils treated with BAL derived from patients with chronic inflammation, and neutrophils treated with a control cytokine GM-CSF.

### Example 8

Antisense Treatment Specifically Decreases Survivin Gene Expression in TF1 Cells and Blocks the GM-CSF- and G-CSF-Mediated Delay of Apoptosis in Neutrophils.

Oligonucleotides

The 20-mer first generation antisense oligonucleotide 4003 targeting the survivin mRNA has been described above. Phosphorothioate (PT) oligos are synthesised by Oswel; all 20 nucleotides are PT analogues: 2OPT. Methylphosphonate (MeP) gapmer oligos are synthesised by Eurogentec (the first and last 5 nucleotides are MeP analogues and the middle 10 are PT analogues (5MeP-10PT-5MeP). Locked nucleic acid (LNA) oligos are synthesised by PrOligo (4LNA-12PT-4LNA).

Cell Culture

TF1 cells are cultured in RPMI 1640 containing 7% conditioned media from recombinant hGM-CSF-expressing WT293 cells and 10% FCS. 4.2×10⁵ TF1 cells are transfected with 4 μg of antisense or missense survivin oligos in each well of a 24 well plate using 8 μl DMRIE reagent (Invitrogen #10459-014) according to the manufacturer's recommendations. Because of the sensititvity of TF1 cells to GMCSF withdrawal, recombinant GMCSF is added to the OPTI-MEM media during transfection. 5 hours after adding the siRNA to the TF1 cells, the media is replaced by RPMI 1640 containing GMSCF and 10% FCS and incubated at 37° C. The cells are harvested 19 hours later by spinning at 1,300 g for 10 min.

Determination of Neutrophil Apoptosis

Neutrophil apoptosis is assessed by oligonucleosomal DNA fragmentation (Dibbert et al.(1999) Proc. Natl. Acad. Sci. USA 96, 13330-13335; Hebestreit et al. (1998), J. Exp. Med. 187, 415-425) according to Nicoletti et al. ((1991) J. Immunol. Meth. 139, 271-279). Briefly, cells are resuspended in hypotonic fluorochrome solution containing 50 μg/ml propidium iodide (Sigma Buchs, Switzerland), 0.1% sodium citrate (Sigma) and 0.1% (vol/vol) Triton-X 100 (Sigma), incubated at 4° C. for 10 h, and analyzed by flow cytometry (FACScan, Becton Dickinson, Heidelberg, Germany).

Real-Time PCR

RNA is isolated using Qiagen's RNeasy Miniprep columns (Qiagen#74104) after lysis on QIAshredder columns (Qiagen #79654). This procedure also involves a DNAse treatment. The RNA is quantified by spectrophotometric analysis and 1 μg is reverse transcribed into cDNA using SuperScriptII RNAseH-Reverse Transcriptase (Invitrogen #18064-014) using oligodT primer according to the manufacturer's instructions. Q-PCR primers amplify a PCR product of 205 nucleotides in length. Forward primer: 1-atgggtgccccgacgttgc-19; Reverse primer:201-ctcccagccttccagctccttg-183. Primers are synthesised by MWG Biotech. Real-time monitoring of PCR amplification of survivin cDNA is performed with TF1 cDNA as previously described (Olie et al.). Relative quantification of survivin expression is performed as described (Olie et al.) utilizing RPS13 as an internal standard. Normalised values of Survivin template are estimated for each sample and these are expressed as a percentage of survivin levels in the mock transfected cells.

FIG. 15 shows real-time PCR quantification of survivin mRNA in TF1 cells transfected with survivin antisense and missense PT, LNA and MeP oligos. The amount of survivin transcript is expressed as a percentage of survivin detected in mock-transfected cells. All three chemistries of survivin-antisense, in contrast to mismatch control oligonucleotides (missense), reduce survivin mRNA levels 19 hours after a 5 hour transfection incubation. PT and LNA antisense are more effective at reducing the survivin mRNA (35% reduction) than the MeP antisense (23% reduction).

FIG. 16 shows a reduction of survivin expression by specific antisense treatment blocks the GM-CSF-mediated delay of apoptosis in neutrophils treated with oligonucleotides having LNA-modified chemistries. Antisense LNA oligonucleotides (closed box), targeted against survivin mRNA, increases spontaneous apoptosis of neutrophils cultured in the presence of GM-CSF assayed 18-h post transfection when compared to neutrophils transfected with mis-sense LNA oligonucleotides (open box). Neutrophils are transfected with oligonucleotides using DMRIE-C reagent as described above and neutrophils apoptosis is assessed by oligonucleosomal DNA fragmentation. Neutrophil apoptosis increases in a dose-dependant manner with antisense LNA oligonucleotide.

### Example 9

Antisense Treatment Specifically Prevents Increases in Survivin Gene Expression in Neutrophils upon Stimulation with Hematopoietins.

Oligonucleotides

The 20-mer first generation antisense oligonucleotide 4003 targeting the survivin mRNA has been described above. The second generation 2′-O-methoxy-ethoxy (2′-MOE) gapmer version of 4003 (as) and its 3-base mismatch sequence control (ms) are synthesized using an Applied Biosystems 394B automated DNA synthesizer as described (De Mesaeker et al. (1995) Acc. Chem. Res. 28, 366-374). Sequences are as follows: antisense survivin, cscscsasgsCsCsTsTsCsCsAsGsCsTscscststsg; mismatch survivin, cscstsasgsCsCsTsTsCsCsAsGsGsTscscstsasg. Small letters refer to 2′-O-(2-methoxy)-ethyl-modified nucleotides, capital letters to DNA, and “s” refers to phosphorothioate linkages. Cells isolated and cultured as described above are treated with survivin antisense oligonucleotides (as) or survivin mismatch control oligonucleotides (ms) at the indicated concentrations. In the experiments in which GM-CSF (50 ng/ml; kind gift of T. Hartung, University of Konstanz, Konstanz, Germany) or G-CSF (25 ng/ml; PeproTech, BioConcept, Allschwil, Switzerland) are added, the oligonucleotides are given 2 h before addition of cytokines.

FIG. 17A shows real-time PCR quantification of survivin mRNA, performed as described above. The lung cancer cell line A549 serves as a standard (=100%). 2-MOE modified oligonucleotides (ms and as) have only little effects on survivin mRNA expression in the absence of survival cytokines following a 4-h transfection period (upper panel). Survivin-antisense (as), in contrast to mismatch control oligonucleotides (ms), prevents increases in survivin mRNA expression upon GM-CSF (middle panel) and G-CSF (lower panel) stimulation in a dose-dependent manner. In case of GM-CSF, 1.0 μM survivin-antisense is sufficient to significantly decrease survivin mRNA levels. In case of G-CSF, the required concentration is 2.5 μM. Values are means ±S.E.M. of three independent experiments. *, p<0.05; **, p<0.01; ***, p<0.001. Statistical analysis is performed by using Student's t test. A p value of <0.05 is considered statistically significant. Mean levels are presented together with standard errors of the mean (S.E.M.). Antisense and mismatch survivin-treated cells are compared at each indicated concentrations.

FIG. 17B shows protein expression of survivin in normal neutrophils is almost not affected by incubation with 2-MOE modified survivin antisense oligonucleotides (as) following a 15-h transfection period (upper panel). However, survivin-antisense, in contrast to 2-MOE modified mismatch control oligonucleotides (ms), prevents increases in survivin expression upon GM-CSF (middle panel) and G-CSF (lower panel) stimulation in a dose-dependent manner. The optimal concentrations of survivin-antisense to block GM-CSF- and G-CSF-increases in survivin protein expression are 2.5 μM and 5 μM, respectively. β-actin immunoblotting is performed to demonstrate equal loading. Results are representative of three independent experiments.

### Example 10

Reduction of Survivin Expression by Specific Antisense Treatment Completely Blocks the GM-CSF- and G-CSF-Mediated Delay of Apoptosis in Neutrophils.

FIG. 18 shows that 2-MOE modified antisense oligonucleotides targeting survivin mRNA (as, closed symbols) increase spontaneous apoptosis of neutrophils after a 10-h transfection period, as assessed by formation of oligonucleosomal DNA-fragments (left panel). GM-CSF (middle panel) and G-CSF (right panel) prevent DNA-fragmentation and survivin-antisense blocked GM-CSF- and G-CSF-mediated survival in a dose-dependent manner. Compared to the unspecific effect of a 3 base-pair mismatch control oligonucleotide (ms; open symbols), the optimal concentrations of survivin-antisense to block GM-CSF- and G-CSF-mediated antiapoptosis are 2.5 μM and 5 μM, respectively. Values are means ±S.E.M. of four independent experiments. *, p<0.05; **, p<0.01; ***, p<0.001.

### Example 11

Survivin-Antisense Treatment Abolishes the Inhibitory Effect of Neutrophil Hematopoietins on Caspase-3 Activation in Neutrophils.

Immunoblotting is carried out as described above using supernatants of cell lysates. Caspase-3 is detected using a polyclonal anti-caspase-3 antibody (1/1000; Becton Dickinson Pharmingen, Heidelberg, Germany))

FIG. 19 A shows apoptosis is associated with decreased levels of the 32-kDa-proform of caspase-3 and with the occurrence of a 17-kDa caspase-3 fragment. Neutrophils cultured in the presence of GM-CSF or G-CSF for 10 h maintain greater amounts of the caspase-3 proform and have slightly decreased levels of the 1 7-kDa fragment compared to untreated or oligonucleotide-treated cells. 2-MOE modified survivin-antisense treatment (as) but not mismatch control oligonucleotides (ms) increase caspase-3 processing in the presence of GM-CSF or G-CSF in a dose-dependent manner. The optimal concentrations of survivin-antisense to block GM-CSF- and G-CSF-mediated inhibitory effects on caspase-3 processing are 2.5 μM and 5 μM, respectively. The same results are obtained in two additional experiments.

Enzymatic Caspase Assay

Neutrophils (2.5×10⁶) are cultured in the presence or absence of GM-CSF (50 ng/ml), G-CSF (25 ng/ml), 2-MOE modified antisense or mismatch survivin oligonucleotides at the indicated concentrations for 10 h, washed with cold PBS and subsequently lysed in 50 μl cell lysis buffer (50 mM HEPES, pH 7.4/0.1% Chaps/5 mM DTT/0.1 mM EDTA) using a Teflon glass homogenizer (VWR International, Ismaning, Germany) on ice for 10 min. After a 10-min centrifugation step at 10,000×g at 4° C., an aliquot of the supernatant is saved for caspase-3 immunoblotting and caspase 3-like activity is measured in 10 μl supernatants as enzymatic-conversion of the colorimetric substrate Ac-DEVD-pNA at 405 nm according to the manufacturer's instructions (QuantiZyme caspase 3 cellular activity assay kit, Biomol, Plymouth Meeting, Pa., USA).

FIG. 19B shows the results of a Caspase-3 activity assay. Increases in the enzymatic activity are detectable in neutrophils undergoing spontaneous apoptosis compared to GM-CSF- or G-CSF-treated cells. Survivin-antisense (as, closed symbols) but not mismatch control oligonucleotides (ms; open symbols) increase caspase-3-like enzymatic activity in the presence of GM-CSF or G-CSF in a dose-dependent manner. The optimal concentrations of survivin-antisense, at which the inhibitory effects of GM-CSF and G-CSF on caspase-3 activity are completely blocked, are 2.5 μM and 5 μM, respectively. Results of two independent experiments are shown (circle, experiment 1; triangle, experiment 2).

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

Amendments to the Specification:

Please replace the paragraph at page 49, from line 1 through line 11, with the following paragraph:

Neutrophils were purified as described in Example 1. RNA was prepared from neutrophils treated with GMCSF. Non-quantitative polymerase chain reaction amplifications were prepared according to standard conditions using primers

SEQ ID NO: 2

| | |
|---|---|
| F1 5′-ATGGGTGCCCCGACGTTGC-3′ | (Predicted Tm:59 C) |

SEQ ID NO: 3

| | |
|---|---|
| R1 5′-TCAATCCATGGCAGCCAGCTG-3′ | (Predicted Tm:57 C) |

These primers will amplify fragments of 429 bp (Survivin), 498 bp (Survivin-2B) and 311 bp (Survivin-Lex3). The results of this experiment are presented in FIG. 10, and illustrate that all three isoforms are expressed in neutrophils treated with GMCSF. A control sample derived from HL60 cells is also shown.

Please replace the paragraph at page 53 from line 23 through line 28 with the following paragraph:

Either antisense or missense oligonucleotides are added directly to each well over a range of concentrations between 0 μM to 0 μM.

Sequence for antisense oligonucleotide is:

| | |
|---|---|
| 5′-CCCAgCCTTCCAgCTCCTTg-3′ | (SEQ ID NO:4) |

Sequence for missense (mismatch) oligo-
nucleotide is:

| | |
|---|---|
| 5′-CCTAgCCTTCCAggTCCTAg-3′ | (SEQ ID NO:5)-- |

Please replace the paragraph on page 55, from lines 15 to 26 with the following replacement paragraph:
RNA is isolated using Qiagen's RNeasy Miniprep columns (Qiagen&num;74104) after lysis on QIAshredder columns (Qiagen &num;79654). This procedure also involves a DNAse treatment. The RNA is quantified by spectrophotometric analysis and 1 μg is reverse transcribed into cDNA using SuperScriptII RNAseH-Reverse Transcriptase (Invitrogen #; 18064-014) using oligodT primer according to the manufacturer's instructions. Q-PCR primers amplify a PCR product of 205 nucleotides in length. Forward primer: 1-atgggtgccccgacgttgc-19 (SEQ ID NO: 6); Reverse primer:201-ctcccagccttccagctccttg-183 (SEQ ID NO: 7). Primers are synthesised by MWG Biotech. Real-time monitoring of PCR amplification of survivin cDNA is performed with TF1 cDNA as previously described (Olie et al. ). Relative quantification of survivin expression is performed as described (Olie et al.) utilizing RPS13 as an internal standard. Normalised values of Survivin template are estimated for each sample and these are expressed as a percentage of survivin levels in the mock transfected cells.

Please replace the paragraph on page 56 from lines 22 through 29 with the following paragraph:
The 20-mer first generation antisense oligonucleotide 4003 targeting the survivin mRNA has been described above. The second generation 2′-O-methoxy-ethoxy (2′MOE) gapmer version of 4003 (as) and its 3-base mismatch sequence control (ms) are synthesized using an Applied Biosystems 394B automated DNA synthesizer as described (De Mesaeker et al. (1995) Acc. Chem. Res. 28,366-374). Sequences are as follows: antisense survivin, cscscsasgsCsCsTsTsCsCsAsGsCsTscscststsg (SEQ ID NO: 8); mismatch survivin, cscstsasgsCsCsTsTsCsCsAsGsGsTscscstsasg (SEQ ID NO: 9). Small letters refer to 2′-O-(2-methoxy)-ethyl-modified nucleotides, capital letters to DNA, and “s” refers to phosphorothioate linkages.

1. A method for detecting apoptosis in a myeloid cell comprising detecting a decrease in Survivin activity or expression by detecting a decrease in any one of:
i) a Survivin polypeptide having an amino acid sequence as set out in SEQ ID NO: 1;
ii) a polypeptide having at least 80% homology with i);
iii) a nucleic acid encoding a polypeptide having the sequence set out in i) or ii);
iv) a nucleic acid which hybridises under stringent conditions to the sequence set out in iii); or
v) the complement of iii) or iv).

2. A method for detecting survival in a myeloid cell comprising detecting an increase in Survivin activity or expression by detecting an increase in any one of:
i) a Survivin polypeptide having an amino acid sequence as set out in SEQ ID NO: 1;
ii) a polypeptide having at least 80% homology with i);
iii) a nucleic acid encoding a polypeptide having the sequence set out in i) or ii);
iv) a nucleic acid which hybridises under stringent conditions to the sequence set out in iii); or
v) the complement of iii) or iv).

3. A method of modulating apoptosis in a myeloid cell comprising the step of increasing, decreasing or otherwise altering the functional activity of
i) a Survivin polypeptide having an amino acid sequence as set out in SEQ ID NO: 1;
ii) a polypeptide having at least 80% homology with i);
iii) a nucleic acid encoding a Survivin polypeptide having the sequence set out in i) or ii);
iv) a nucleic acid which hybridises under stringent conditions to the sequence set out in iii); or
v) the complement of iii) or iv).

4. A method as claimed in claim 3 comprising decreasing Survivin gene expression.

5. A method as claimed in claim 3 comprising increasing Survivin gene expression.

6. A method as claimed in any of claims 3 to 5 comprising:
a) providing an expression vector comprising a nucleic acid sequence encoding a Survivin polypeptide, said nucleic acid sequence being selected from the group consisting of:
i) a nucleic acid encoding a Survivin polypeptide having an amino acid sequence as set out in SEQ ID NO: 1;
ii) a nucleic acid which hybridises under stringent conditions to the sequence set out in i); or
iii) the complement of ii);
b) introducing the expression vector into the cell and maintaining the cell under conditions permitting expression of the encoded polypeptide in the cell.

7. A method as claimed in any of claims 1 to 6 wherein the polypeptide is a splice variant of Survivin.

8. A method as claimed in claim 7 wherein the splice variant is selected from the group consisting of Survivin delta ex3 and Survivin 2B.

9. A method as claimed in claim 4 wherein a decrease in Survivin gene expression is effected by antisense.

10. A method as claimed in any of claims 1 to 9 wherein the myeloid cell is a neutrophil.

11. A method of treatment of inflammatory disease comprising administering a modulator of Survivin gene expression or functional activity.

12. A method as claimed in claim 11 wherein the modulator is an antisense RNA molecule of Survivin gene expression.

13. Use of a modulator of Survivin gene expression or functional activity in the manufacture of a medicament for use in the treatment of inflammatory disease.

14. A use as claimed in claim 13 wherein said modulator is an antisense RNA molecule modulator of Survivin gene expression.


SEQUENCE LISTING (9 sequences)

SEQ ID NO: 1 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 2 - DNA, 19 bp, Artificial
  Forward survivin primer
atgggtgccccgacgttgc

SEQ ID NO: 3 - DNA, 21 bp, Artificial
  Reverse survivin primer
tcaatccatggcagccagctg

SEQ ID NO: 4 - DNA, 20 bp, Artificial
  Survivin antisense oligonucleotide
cccagccttccagctccttg

SEQ ID NO: 5 - DNA, 20 bp, Artificial
  Survivin missense (mismatch) oligonucleotide
cctagccttccaggtcctag

SEQ ID NO: 6 - DNA, 19 bp, Artificial
  Forward quantitative PCR primer
atgggtgccccgacgttgc

SEQ ID NO: 7 - DNA, 22 bp, Artificial
  Reverse quantitative PCR primer
ctcccagccttccagctccttg

SEQ ID NO: 8 - DNA, 20 bp, Artificial
  2′-O-methoxy-ethoxy version of Survivin antisense oligonucleotide
cccagccttccagctccttg

SEQ ID NO: 9 - DNA, 20 bp, Artificial
  2′-O-methoxy-ethoxy version of Survivin mismatch oligonucleotide
cctagccttccaggtcctag